(19) United States
(12) Patent Application Publication
(10) Pub. No.: US 2016/0361496 A1
(43) Pub. Date: Dec. 15, 2016

(54) AUTOINJECTOR

(71) Applicant: Nuance Designs of CT, LLC, Woodbridge, CT (US)

(72) Inventors: Carlos Guillermo, Atascadero, CA (US); David DeSalvo, Lake Hiawatha, NJ (US)

(73) Assignee: Nuance Designs of CT, LLC, Woodbridge, CT (US)

(21) Appl. No.: 15/183,533

(22) Filed: Jun. 15, 2016

Related U.S. Application Data
(60) Provisional application No. 62/175,755, filed on Jun. 15, 2015.

(51) Int. Cl. A61M 5/20; A61M 5/32

(52) U.S. Cl. CPC A61M 5/2046; A61M 5/3202; A61M 2205/583; A61M 2205/6018

(57) ABSTRACT

An autoinjector that includes a canister having a liquefied gas, a flow regulator, and an actuator is provided. The flow regulator engages the canister to receive a flow of gas generated by the liquefied gas. The actuator is coupled to the canister and the flow regulator, and includes a passageway for receiving the flow of gas downstream from the flow regulator. The actuator moves relative to the canister between an engaged position whereby the flow regulator is in fluid communication with the canister for receiving the flow of gas and a storage position whereby the flow regulator is spaced from the canister.

### CROSS-REFERENCE TO RELATED APPLICATIONS

This application claims the benefit of U.S. Provisional Application No. 62/175,755, filed Jun. 15, 2015, entitled “AUTOINJECTOR” the entire disclosure of which is incorporated by reference herein in its entirety.

### BACKGROUND OF THE INVENTION

The present invention relates generally to drug delivery devices. Specifically, the present invention relates to an autoinjector for delivering a drug through a needle-based drug delivery device.

An autoinjector is a medical device designed to deliver one or more doses of a particular drug in a manner that facilitates self-administration of the drug via a syringe needle. Autoinjectors were originally designed for military use to counteract nerve-agent poisonings. The devices later moved into the civilian realm, with the first civilian devices being introduced in the mid to late 1970s, to dispense epinephrine to treat anaphylaxis. More recently, these devices have seen broadened use.

By design, autoinjectors are easy to use and are intended for administration by patients to themselves, or by untrained personnel. Thus, they are typically self-contained and designed to require only a few basic steps to operate.

Typically, autoinjectors are spring actuated. This means that one or more springs are used to drive the drug through the needle of the autoinjector, and in some cases, to insert the needle into the patient as well. At least one spring is typically used to apply a force to a stopper of a syringe or cartridge, much in the manner that a person would manually actuate a syringe plunger, and drive the drug out of the syringe into the injection site. These autoinjectors typically deliver a full dose of their drug in about 5 to 10 seconds.

An alternative form of autoinjector is the gas jet injector, which dispenses with a needle entirely; instead using a high-pressure narrow jet of the drug itself to penetrate the skin. Gas jet injectors have predominantly been used for mass vaccinations, not single dose delivery, and involve delivery of the drug at pressures of about 4,000 psi almost instantaneously. Newer gas jet injectors use slightly lower pressures. In general however, gas jet injectors are limited in volume they can deliver in a single “shot” and the depth to which they can deliver the drug. In addition, as an explosive/high impact technology, they cause impact and jarring that can be problematic.

Current designs in spring actuated autoinjectors involve making tradeoffs among various controllable and uncontrollable factors to insure reliable, proper and complete dose delivery. However, the selected tradeoffs that provide for reliable, proper and complete dose delivery can result in the inability to provide certain desirable feature(s) or requiring of greater complexity to provide less than desirable version(s) of such feature(s).

### BRIEF SUMMARY OF THE INVENTION

In accordance with a preferred embodiment, the present invention provides an autoinjector that includes a canister, a flow regulator, and an actuator. The canister includes a liquefied gas. The flow regulator engages the canister to receive a flow of gas generated by the liquefied gas. The actuator is coupled to the canister and the flow regulator. The actuator includes a passageway for receiving the flow of gas downstream from the flow regulator. The actuator moves relative to the canister between an engaged position whereby the flow regulator is in fluid communication with the canister for receiving the flow of gas and a storage position whereby the flow regulator is spaced from the canister. The actuator further includes an exhaust passageway about a distal end of the actuator, a seal sealingly engaging the exhaust passageway when the actuator is in both the engaged and storage positions, and a seal between the canister and the flow regulator. The seal is between the canister and the actuator and the flow regulator includes a piercing member for engaging the canister. The flow regulator includes a porous member for receiving the flow of the gas.

In accordance with another preferred embodiment, the present invention provides an autoinjector that includes a canister and an actuator. The canister includes a liquefied gas for delivering a flow of gas. The actuator is coupled to the canister and includes a housing body, a through hole through the housing body for receiving the flow of gas, and a flow regulation device adjacent the through hole. The actuator moves relative to the canister between an engaged position whereby the canister engages the actuator to allow fluid communication of the flow of gas with the through hole, and a storage position whereby the canister is disengaged with the actuator and the through hole is not in fluid communication with the flow of gas. The actuator also includes a hub assembly having a piercing member e.g., a stake, a spike, a cannula or other sharp structure. The hub assembly engages the canister in the engaged position and is spaced from the canister in the disengaged position.

In accordance with yet another preferred embodiment, the present invention provides an actuator assembly for an autoinjector that includes an actuator, an activator and a biasing member. The actuator includes a cam surface. The activator circumscribes the actuator and is rotatable about the actuator between an initial position wherein a distal end of the actuator is spaced from a distal end of the activator at a first distance, and an activated position wherein the distal end of the actuator is spaced from the distal end of the activator at a second distance. The biasing member biases the activator and actuator relative to each other. The biasing member biases one of the actuator and activator to move between the initial position and the activated position upon engagement of the cam surface. The actuator assembly further comprising an activator housing having a tab, and wherein the activator includes a track for receiving the tab. The track includes an axial track portion and a circumferential track portion. The track also includes a first portion for engaging the tab when the activator is in the initial position, and a second end circumferentially spaced from the first end for engaging the tab when the activator is in the activated position. The biasing member provides a rotational biasing force and an axial biasing force. The biasing member further biases one of the actuator and activator to move to a final position whereby the activator engages a locking feature of the actuator.

In accordance with another preferred embodiment, the present invention provides an actuator assembly for an autoinjector that includes an actuator, an activator, and an actuator housing. The activator includes a track. The activator circumscribes the actuator and is moveable relative to the actuator between an initial position wherein a distal end of the actuator is spaced from a distal end of the activator at a first distance, an activated position wherein the distal end of the actuator is spaced from the distal end of the activator at a second distance, and a retracted position wherein the distal end of the actuator is spaced from the distal end of the activator at a third distance. The actuator housing is coupled to the activator and includes a tab for engaging the track. The tab directly engages the track to releasably maintain the position of the actuator relative to the activator in the initial position, activated position or retracted position.

In accordance with yet another preferred embodiment, the present invention provides an autoinjector that includes a shield and an actuator assembly. The shield housing a syringe moveable relative to the shield. The actuator assembly delivers a driving force that operatively engages the syringe. The actuator assembly includes an actuator, an activator coupled to the actuator and movable relative to the actuator between a first position wherein the activator is stationary with respect to the actuator, a second position circumferentially spaced from the first position, and a third position circumferentially spaced from the first and second positions, and a biasing member biasing the activator and actuator relative to each other to move one of the activator and actuator between first, second and third positions. The second position is circumferential spaced from the first position in a first direction, and the third position is circumferential spaced from the first position in the first direction and axially spaced from the second position. The second position is axially spaced from the first position. The biasing member biases one of the actuator and activator in both an axial direction and a rotational direction. The activator further includes an activator cam surface. The shield includes a proximally extending latch member, and the shield moves between an initial position covering a distal end of the syringe and an injection position, and when moving from the initial position to the injection position the proximally extending latch member engages the activator cam surface to move the activator from the first position to the second position.

In accordance with another preferred embodiment, the present invention provides an autoinjector that includes a housing, a syringe, and an actuator assembly. The syringe is housed within the housing and includes a syringe barrel. The actuator assembly includes a canister having a liquefied gas, a flow regulator for engaging the canister to receive a flow of gas generated by the liquefied gas, and an actuator housed within the syringe barrel. The actuator includes a passageway for receiving the flow of gas downstream from the flow regulator. The actuator moves relative to the canister between an engaged position whereby the flow regulator engages the canister and a storage position whereby the flow regulator is spaced from the canister. The actuator further includes an exhaust passageway about a distal end of the actuator. The autoinjector further includes a seal between the syringe barrel and the actuator. The seal is movable between a first position sealingly engaging the exhaust passageway and a second position allowing the flow of gas through the exhaust passageway.

In accordance with yet another preferred embodiment, the present invention provides an autoinjector that includes a housing, a syringe, and an actuator assembly. The syringe is housed within the housing and includes a syringe barrel and a piston slidably engaged with the syringe barrel. The actuator assembly includes a canister having a liquefied gas at a vapor pressure of P1, a flow regulator for engaging the canister to receive a flow of gas generated by the liquefied gas at a pressure of P1, and an actuator coupled to the syringe barrel. The flow regulator moves relative to the canister between an engaged position whereby the flow regulator engages the canister and a storage position whereby the flow regulator is spaced from the canister. The actuator includes a passageway and when the flow regulator is moved to the engaged position the passageway receives the flow of gas downstream from the flow regulator and discharges the flow of gas into the syringe barrel at a pressure P2 that is less than P1 to drive the piston distally along the syringe barrel. The pressure within the syringe barrel is substantially maintained at P2 for driving the piston to be fully seated against a distal end of the syringe barrel. The autoinjector further comprising a seal between the syringe barrel and the actuator. The actuator further includes an exhaust passageway about a distal end of the actuator in fluid communication with the flow regulator. The seal is movable between a first position sealingly engaging the exhaust and a second position allowing the flow of gas through the exhaust passageway. After the piston fully seats against the distal end of the syringe barrel, the pressure within the syringe barrel increases to P3, which is greater than P2 and less than P1, until the pressure within the syringe barrel moves the seal from the first position to the second position. The piston is driven distally along the syringe barrel at a substantially constant speed by the pressure P2, and when the speed of travel of the piston decreases, the pressure within the syringe barrel increases from P2 to P2′.

In accordance with another preferred embodiment, the present invention provides an autoinjector that includes a shield, a cradle assembly, a housing member, and a biasing member. The cradle assembly is coupled to a medicine cartridge and slidably attached to the shield. The housing member houses the cradle assembly, wherein the cradle assembly is moveable between a primary position, a secondary position, and a tertiary position relative to the shield. The biasing member is coupled to the cradle assembly and shield for biasing the cradle assembly. The cradle assembly includes a distal body and a proximal body slidably coupled to each other. The distal body and proximal body are moveable from the first position to the second position. The proximal body is moveable from the second position to the third position relative to the distal body. The biasing member biases the proximal body. The autoinjector further includes cooperating catches on the distal body of the cradle assembly and shield for engaging one another to hold the distal body in the second position.

In accordance with yet another preferred embodiment, the present invention provides an autoinjector that includes a housing, a syringe, and an actuator. The syringe includes a syringe barrel housed within the housing. The actuator is partially housed within the syringe barrel and defines an interior volume within the syringe barrel, the actuator including a through hole about a distal end. The interior volume is in fluid communication with the through hole and a passageway extending from the through hole to an exterior of the housing. The passageway includes an exhaust passageway about a distal end of the actuator. The passageway further includes a spacing between a seal positioned within the syringe barrel and the actuator. The seal is moveable between a first position sealingly engaging the exhaust passageway, and a second position spaced from the exhaust passageway defining the spacing between the seal and the actuator.

In accordance with another preferred embodiment, the present invention provides an autoinjector that includes a housing, a medicine cartridge, and an adaptive force. The medicine cartridge includes a chamber storing a medicament and a piston operable to drive the medicament from the chamber, wherein the medicine cartridge is housed within the housing. The adaptive force is applied to the piston to drive the medicament from the chamber, wherein the adaptive force increases or decreases based upon a change in speed of travel of the piston. The autoinjector further comprising an actuator assembly operatively connected to the medicine cartridge that includes a canister having a liquefied gas for providing the adaptive force, and a flow regulator for releasing the adaptive force from the canister. A flow of gas provided by the liquefied gas exits the canister and applies an adaptive force to the piston in a controlled manner such that: i) the adaptive force will be constant at a constant injection rate, ii) the adaptive force will increase, if the injection rate slows to below the constant injection rate, and iii) the adaptive force will decrease, as the injection rate increases from below the constant injection rate towards the constant injection rate.

### BRIEF DESCRIPTION OF THE SEVERAL VIEWS OF THE DRAWINGS

The foregoing summary, as well as the following detailed description of the preferred embodiments of the invention, will be better understood when read in conjunction with the appended drawings. For the purpose of illustrating the invention, there are shown in the drawings preferred embodiments of the invention. It should be understood, however, that the invention is not limited to the precise arrangements and instrumentalities shown.

In the drawings:

FIG. 1 is a perspective view of an autoinjector in accordance with a preferred embodiment of the present invention;

FIG. 2 is a longitudinal cross-sectional view of the autoinjector of FIG. 1 taken along a midline plane;

FIG. 3 is another longitudinal cross-sectional view of the autoinjector of FIG. 1 taking along a plane transverse to the midline plane of FIG. 2;

FIG. 4 is an enlarged partial cross-sectional view of a proximal end of the autoinjector shown in FIG. 3;

FIG. 4A is a greatly enlarged partial cross-sectional view of a proximal end of the autoinjector shown in FIG. 3;

FIG. 5 is a perspective view of the autoinjector of FIG. 1 with a housing and a shield component omitted for purposes of illustration;

FIG. 6 is a perspective view of the autoinjector of FIG. 1 in an initial armed state with a housing component omitted;

FIG. 7 is a perspective view of the autoinjector of FIG. 1 in a retracted state with a housing component omitted;

FIG. 8 is a perspective view of a shield of the autoinjector of FIG. 1;

FIG. 9 is a perspective view of an actuator of the autoinjector of FIG. 1;

FIG. 10 is a perspective view of a flow regulator of the autoinjector of FIG. 1;

FIG. 11 is a bottom perspective view of the flow regulator of FIG. 10;

FIG. 12 is a partial side perspective view of the autoinjector of FIG. 1 with certain components omitted for purposes of illustration;

FIG. 12A is a partial perspective view of a proximal end of the autoinjector of FIG. 1 in an activated or needle insertion state with certain components omitted and in phantom for purposes of illustration;

FIG. 12B is a partial perspective view of a proximal end of the autoinjector of FIG. 1 in a retracted state with certain components omitted and in phantom for purposes of illustration;

FIG. 13 is a perspective view of an activator of the autoinjector of FIG. 1;

FIG. 13A is another perspective view of the activator of FIG. 13;

FIG. 14 is a top plan view of the activator of FIG. 13;

FIG. 15 is a perspective view of a cap of the autoinjector FIG. 1;

FIG. 16 is a top perspective view of the cap of FIG. 15;

FIG. 17 is a bottom plan view of the cap of FIG. 15;

FIG. 18 is a partial perspective view of a proximal end of the autoinjector of FIG. 1 with certain components omitted;

FIG. 19 is another partial perspective view of a proximal end of the autoinjector of FIG. 1 with certain components omitted;

FIG. 20 is a perspective view of the autoinjector of FIG. 1 with a distal section of the housing removed and in a ready to use state;

FIG. 21 is a longitudinal cross-sectional view of the autoinjector of FIG. 1 in an activated state;

FIG. 22 is a longitudinal cross-sectional view of the autoinjector of FIG. 1 in a needle insertion state;

FIG. 23 is a longitudinal cross-sectional view of the autoinjector of FIG. 1 in an injecting state;

FIG. 24 is a longitudinal cross-sectional view of the autoinjector of FIG. 1 in an end of dose state;

FIG. 25 is a longitudinal cross-sectional view of the autoinjector of FIG. 1 in a retracted state;

FIG. 26A is a partial anterior view of the autoinjector of FIG. 1 illustrating a visually identifiable feature of the autoinjector in an initial state;

FIG. 26B is a partial anterior view of the autoinjector of FIG. 1 illustrating a visually identifiable feature of the autoinjector in an activated state;

FIG. 26C is a partial anterior view of the autoinjector of FIG. 1 illustrating a visually identifiable feature of the autoinjector in a retracted state;

FIG. 27 is a partial perspective view of a proximal end of the autoinjector of FIG. 1 illustrating a visually identifiable feature in an activated state with certain features drawn in phantom for purposes of illustration.

FIGS. 28A-C are various views of an embodiment of a flow regulator applicable to the present invention;

FIG. 28D is a cross-sectional view of the flow regulator within an actuator of the present invention;

FIGS. 29A-C are various views of another embodiment of a flow regulator applicable to the present invention; and

FIGS. 30A-C are various views of yet another embodiment of a flow regulator applicable to the present invention.

### DETAILED DESCRIPTION OF THE INVENTION

Reference will now be made in detail to the preferred embodiments of the invention illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, above, below and diagonal, are used with respect to the accompanying drawings. Such directional terms used in conjunction with the following description of the drawings should not be construed to limit the scope of the invention in any manner not explicitly set forth. Additionally, the term “a,” as used in the specification, means “at least one.” The terminology includes the words above specifically mentioned, derivatives thereof, and words of similar import.

Certain terminology is used in the following description for convenience only and is not limiting. The words “right,” “left,” “upper,” and “lower” designate directions in the drawings to which reference is made. The terminology includes the words above specifically mentioned, derivatives thereof, and words of similar import. With reference to an autoinjector, the “distal end” of the autoinjector refers to the end of the autoinjector towards the needle while the “proximal end” of the autoinjector refers to the end of the autoinjector towards the indicators or actuator assembly.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the embodiments of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

For ease of explanation, as used herein, the term “syringe” is intended mean any combination of a drug-containing container, a hypodermic needle and a pathway between the two through which the drug can be delivered from the container into a living body via the hypodermic needle, irrespective of the relative proximity between the container and needle themselves. Representative, specific examples of “syringes” as defined herein include (but are not intended to be limited to): conventional staked-in needle syringes, ISO 11040-4 conforming prefilled syringes, removable hub needle/syringe body systems including those with a luer taper, infusion sets, single use and multi-use cartridge-based syringe systems, multi-chambered and variable dose syringes, as well as cartridges, vials and pouches (rigid or collapsible), and a drug to be used in conjunction with a needle to deliver an injection volume (i.e., a dose) of the drug.

Similarly, the use of the term “autoinjector” herein is intended to encompass both the conventional understanding of that term, as well as any other small form factor, hand-holdable or wearable, injection-type, or infusion-type (i.e., for delivery of a drug via a needle over a period of time lasting on the order of several minutes) drug delivery device.

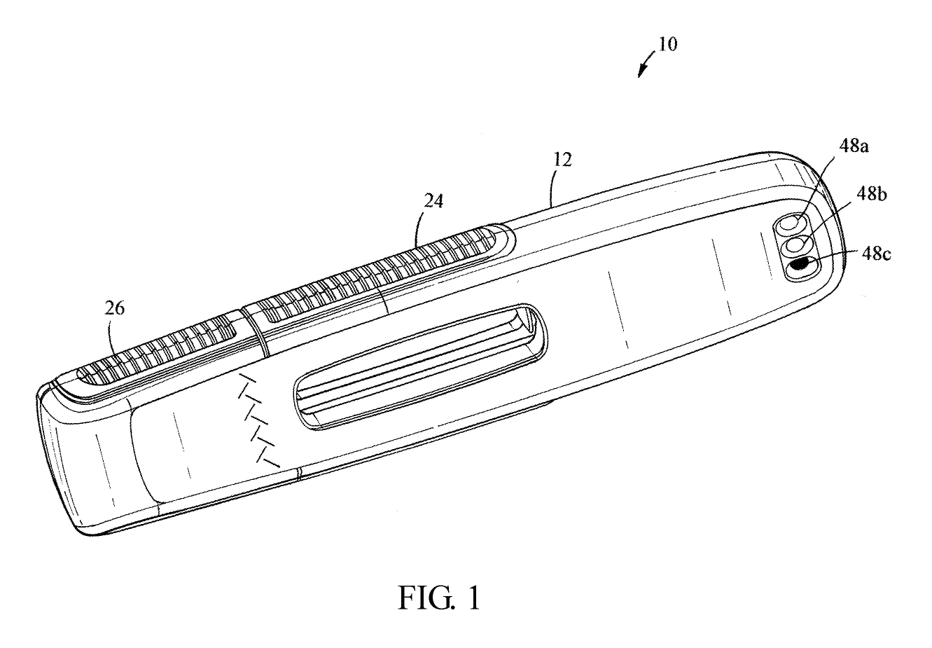

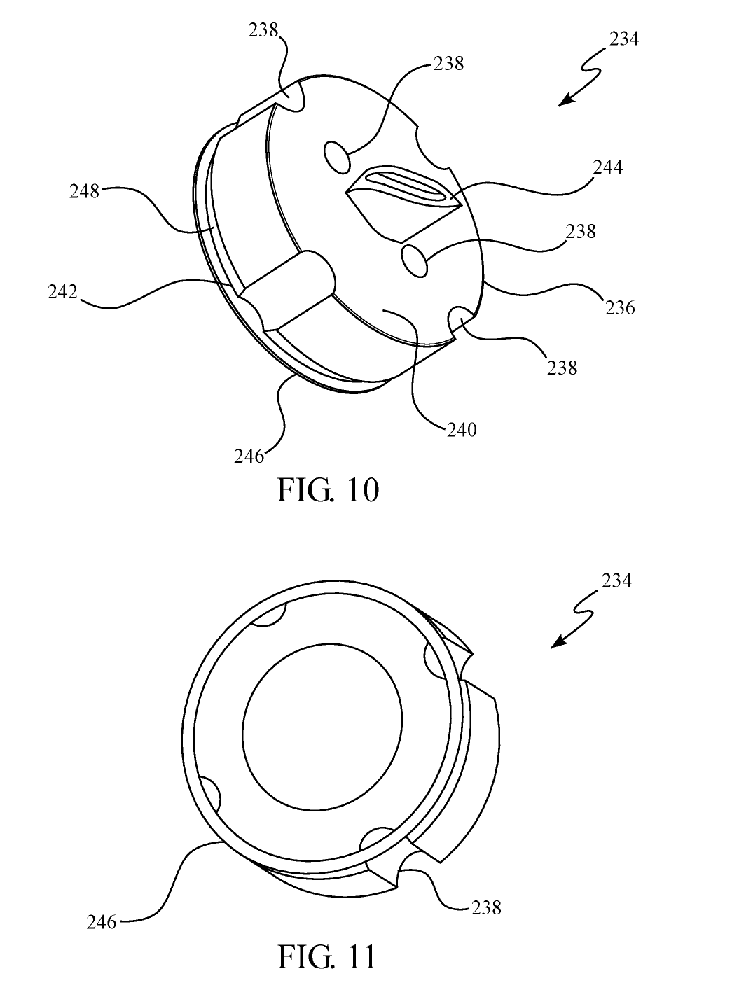

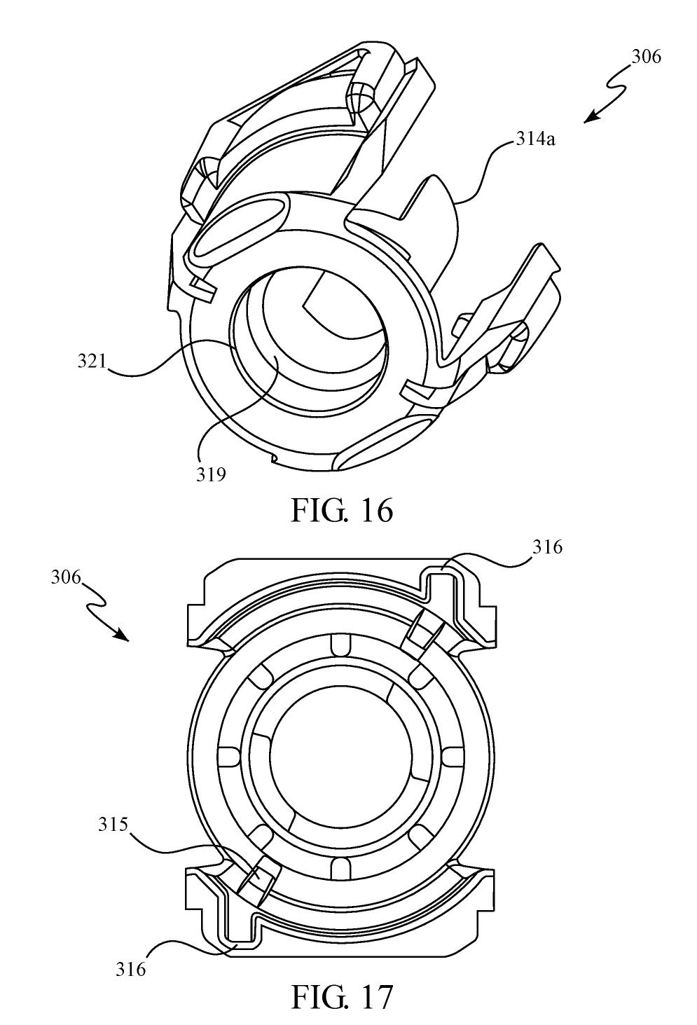

Referring to FIGS. 1-27 there is shown a preferred embodiment of an autoinjector 10 in accordance with the present invention. The autoinjector includes a housing or housing member 12, a syringe 14, and an actuator assembly 200.

The syringe 14 is preferably a staked needle syringe that includes a needle 18, a syringe barrel 20 having a lip 22, and a piston or plunger 25. The syringe 14 is housed within the housing 12 and movable relative to the housing, as further discussed below.

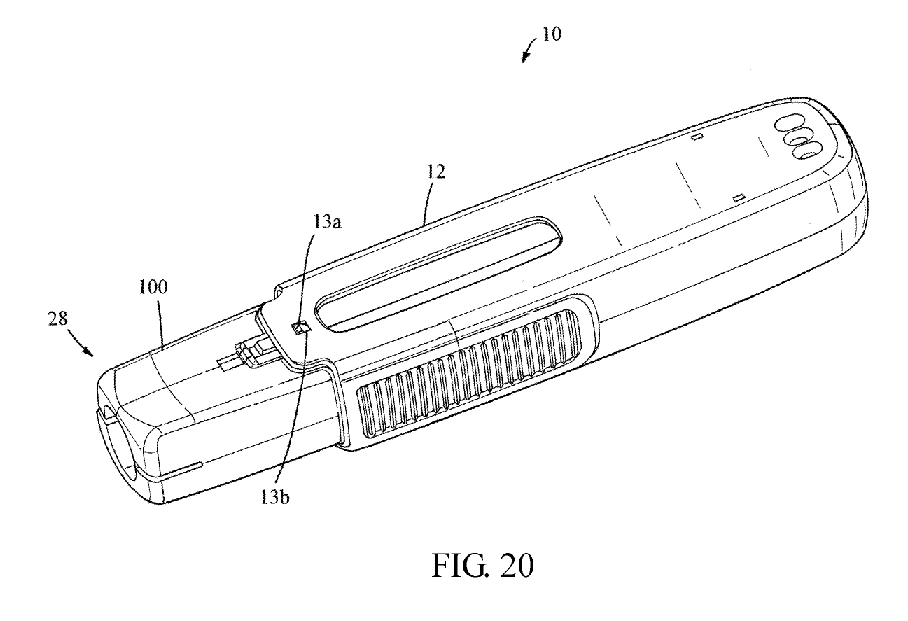

The housing 12 is configured as best shown in FIG. 1. The housing includes a first section 24 to be grasped by a user's hand and a second section 26 about a distal end of the autoinjector. The second section 26 is a removable housing section designed to be removed by the user for exposing the distal end 28 of the autoinjector, as best shown in FIG. 20.

Referring to FIG. 3, mounted within the second section 26 is a pair of gripping members 30 for engaging a needle shield 32 of the syringe. Preferably the gripping members 30 extend inwardly from the walls of the second section housing and are positioned diametrically opposed from each other. Each gripping member also includes a plurality of ridges to facilitate gripping of the needle shield 32, which can include ridges on its outer surface.

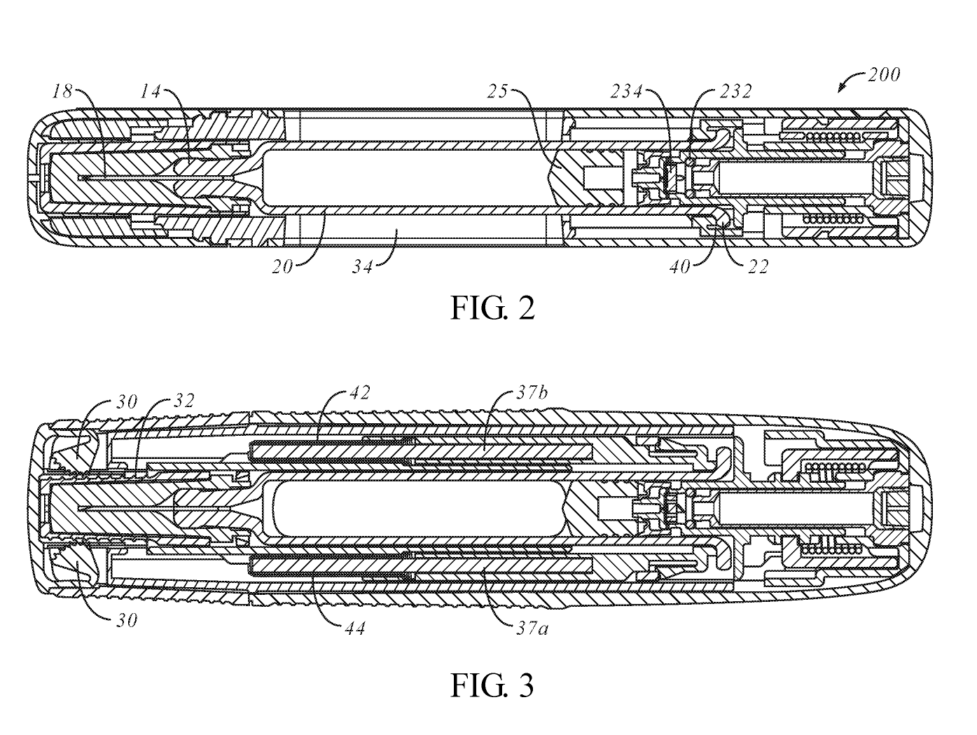

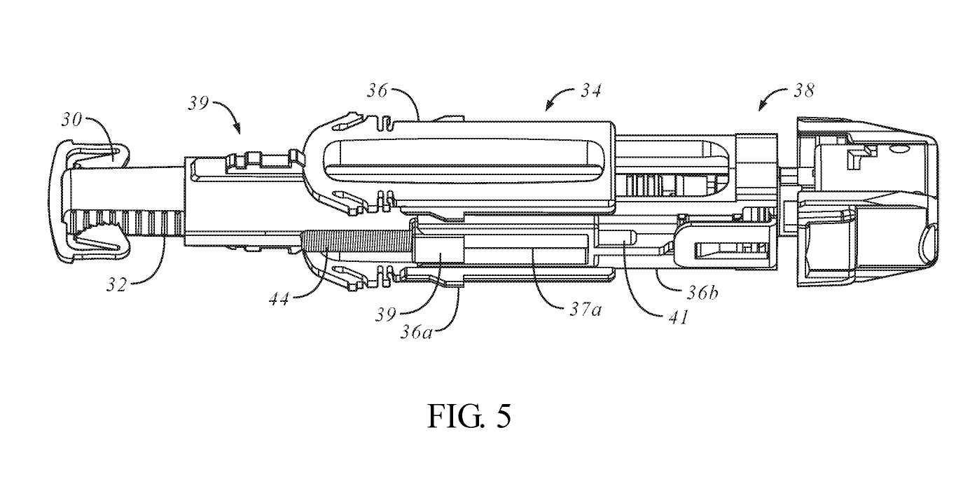

The needle shield 32 can be configured as any conventional needle shield for shielding the syringe needle. The needle shield is releasably mounted to a cradle 34 (FIG. 5) that houses and retains the syringe 14, as further discussed below. FIGS. 2 and 3 are cross-sectional views of the housing 12 assembled to the cradle 34 and autoinjector shield 100. The autoinjector shield 100 houses the syringe and is moveable relative to the syringe. Upon separation of the second section 26 from the first section 24, the gripping members retain the needle shield 32 to withdrawal it from the cradle, as shown in FIG. 20.

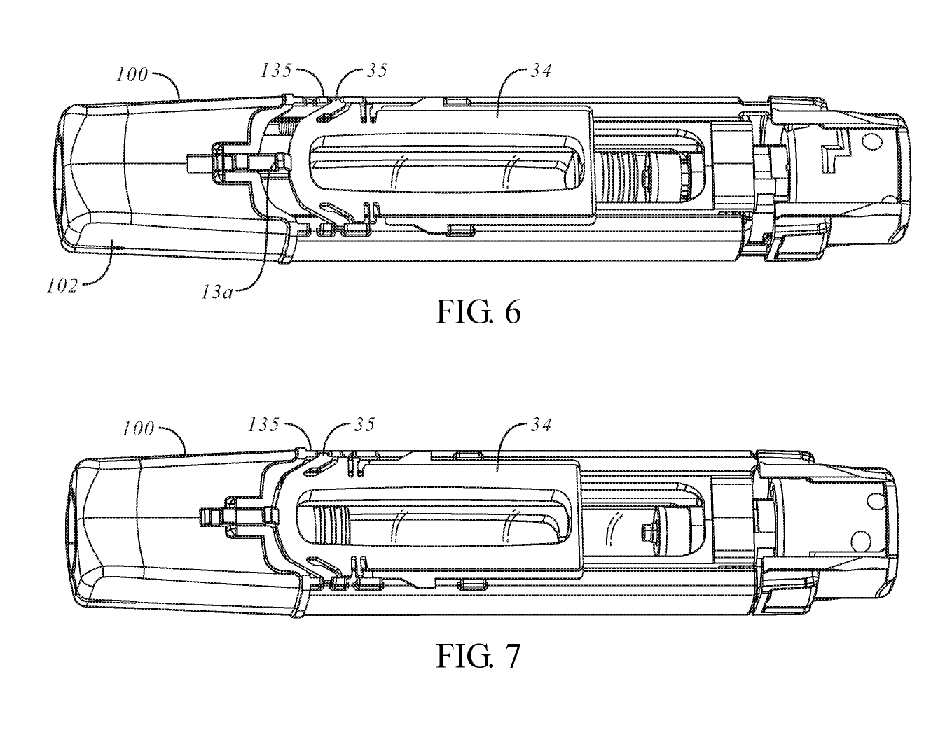

Referring to FIGS. 6 and 20, the housing 12 is connected to the cradle 34 by cooperating detents or catches 13a on the cradle and 13b on the housing.

The cradle 34 is configured as best shown in FIGS. 2, 3, 5 and 6. The cradle 34 is coupled to the syringe or medicine cartridge 14 and movable relative to the shield 100. The cradle 34 includes a two part body 36 having an interior sized sufficiently to house the syringe 14. About a proximal end 38 of the cradle is a recess 40 engaging the lip 22 of the syringe barrel 20. The cradle is also sized to have a longitudinal length sufficient to allow a majority of the needle 18 to extend outwardly from a distal end 39 of the cradle.

The body 36 includes a distal body 36a and a proximal body 36b, as best shown in FIG. 5. Collectively, the distal body, proximal body, and other components is referred to herein as a cradle assembly. The distal body and proximal body are slidably coupled together by a pair of pins 37a, 37b and biasing members 42, 44, about lateral sides of the distal body and proximal body. The proximal body 36b includes the recess 40 for engaging and retaining the syringe 14.

Referring to FIGS. 3 and 5, the distal body 36a of the cradle 34 houses the first and second retraction biasing members 42, 44 positioned along its lateral sides. Each of the first and second retraction biasing members include a proximal end engaged with the distal body 36a about opposite lateral sides of the distal body. Specifically, the distal body 36a include slots 39 about the lateral sides of the distal body. Each slot 39 includes a distal end opening for the passage of a respective biasing member therethrough. Further, each distal end opening is sized to be smaller than a proximal end of the biasing member. That is, each respective biasing member 42, 44 is preferably configured as a compression spring or extension spring having a main body portion of substantially constant diameter, a proximal end having a larger diameter than the main body portion, and a distal end having a diameter smaller than the main body portion. As such, the biasing member is sized and configured to pass through the slot 39 but retained therein as a result of its larger diameter proximal end which is sized to be larger than the distal end opening of the slot 39.

Referring back to FIG. 5, the proximal body 36b includes mounts or recesses 41 about its lateral sides for receiving and holding respective pins 37a, 37b. Preferably, each mount is sized and configured so that a respective pin 37a or 37b is press-fitted therein.

The biasing members 42, 44 and pins 37a, 37b are sized and configured such that the pins are received within the main body portion of the biasing member. Further, the pin diameter is sized to be greater than the distal end of the biasing member so that the pin does not pass through the distal end of the biasing member, but instead contained therein. Thus, as the distal body and proximal body are moved towards each other, each pin forces a respective biasing member to elongate thereby having each respective biasing member apply a counterforce between the distal body and proximal body. As a result, the retraction biasing members biases the proximal body of the cradle 34 in the proximal direction upon completion of an injection by the autoinjector thereby retracting the syringe fully within the shield 100 of the autoinjector.

Referring to FIGS. 6 and 7, the cradle 34 is movably mounted to the shield 100 via cooperating catches 35 on the cradle and cooperating catches 135 on the shield. The cooperating catches 35, 135 releasably engage one another to hold the cradle in the initial position or armed position (FIG. 6) and retracted position (FIG. 7). The cradle is also moveable between extended and retracted positions relative to the shield and housing 12.

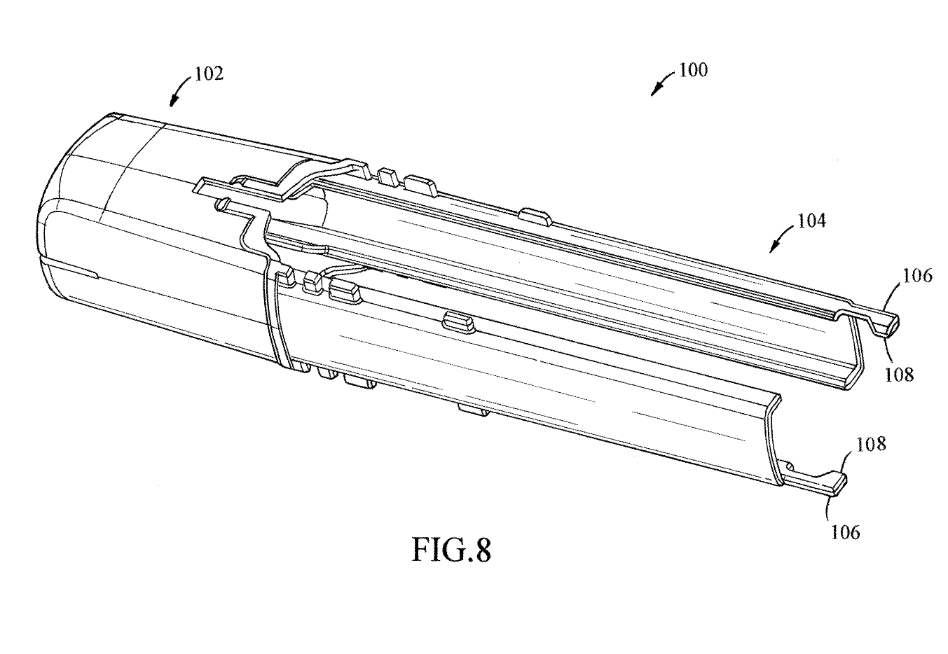

Referring to FIG. 8, the shield 100 includes a distal end 102 for shielding the syringe needle in a retracted state and a proximal end 104. The proximal end 104 is configured as spaced apart slats having an elongated opening extending there between for receipt of the cradle 34. About a most proximal end of this shield is a proximately extending legs or latch member 106. The leg 106 includes an inwardly extending tab 108 for operatively engaging the activator assembly, as further discussed below. Preferably, each slat of the proximal end 104 has an individual leg 106 extending there from such that the proximal end includes a pair of proximately extending legs. More preferably, one of the pair of slats has its individual leg positioned about an anterior side of the slat, while the other of the pair of slats has its individual leg positioned about a posterior side of the slat.

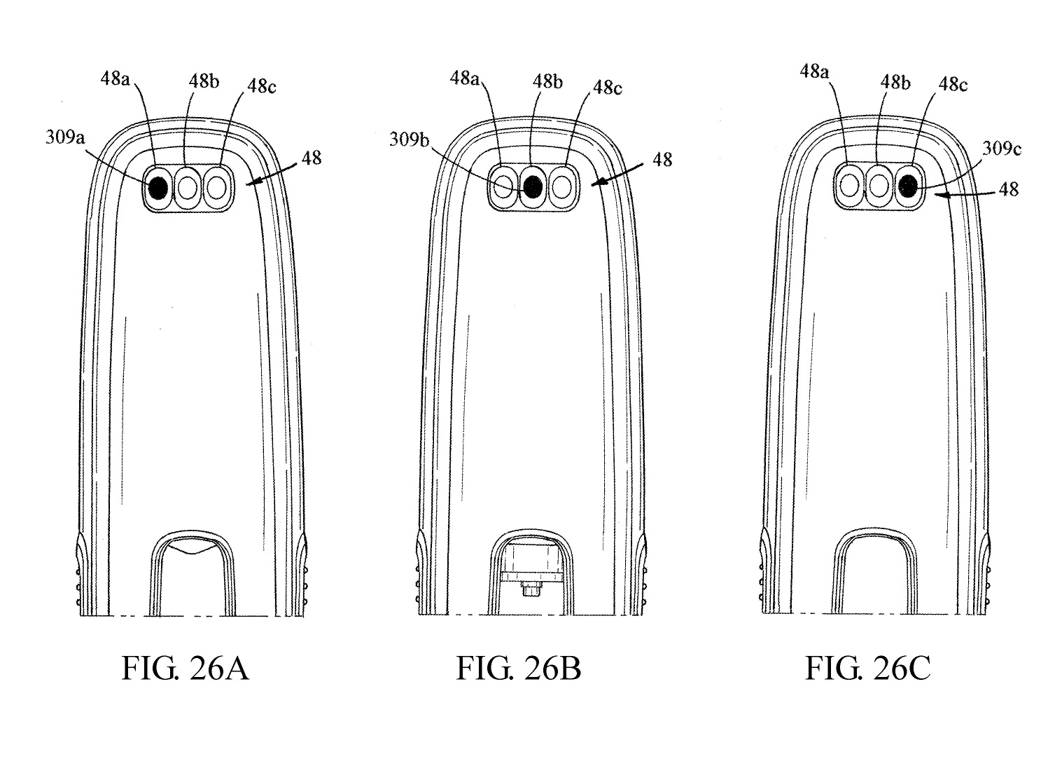

Referring to FIGS. 1 and 26A-C, the first section of the housing 12 also includes a visual indicator system 48 comprising a series of window 48a, 48b, 48c positioned about a proximal end of the housing and along a major face or anterior face of the autoinjector. As further described below, the series of windows provide a visual indication as to the state of operation of the autoinjector. Specifically, each window is activated e.g., by displaying a visually identifiable feature therethrough, based upon the state of operation of the autoinjector. Window 48a is activated when the autoinjector is in the initial or ready to use state (FIG. 26A). Window 48b is activated when the autoinjector is in the activated or needle insertion state (FIG. 26B), and any other state between the initial ready to use state and the retracted state. Window 48c is activated when the autoinjector in the retracted state so as to identify to the user that the syringe needle has completely retracted into the autoinjector (FIG. 26C).

While the visual indicator system 48 preferably includes three windows for indicating respective states of the autoinjector, the visual indicator system can alternatively be formed out of a single window or a plurality of windows e.g., 2, 4, 5 or more than 5 windows, for indicating any of a series of states or changes of state that the autoinjector progresses through from a unused ready for use state to a finished/spent retracted state. Further, the visual indicator system 48 can be configured to include any type of indicator visible through the window(s) e.g., color coded indicators and various symbols or marking to associate with and identify the various states of the autoinjector. As further discussed below, indicators 309a, 309b and 309c illustrate an exemplary type of visual indicator applicable to the present embodiments.

The actuator assembly 200 is best shown in FIGS. 2-7. The actuator assembly 200 includes an activator assembly 300, a canister 204, and an actuator 206. The actuator assembly delivers a driving force that operatively engages the syringe 14 upon actuation of the autoinjector.

The canister 204 is preferably a tubular canister having a tubular body 208, and an openable proximal end sealable by a cap 212, and a distal end 214. The distal end 214 has a neck 216 that is narrower then the tubular body and an opening covered by a pierceable seal 218.

The canister includes a driver 223 e.g., a propellant, or compressed or liquefied gas, that acts as a constant pressure source and is used to apply a force to a component of the actuator assembly or syringe, for example, a stopper, a rod, the cradle or other member, in a controlled manner, to thereby deliver the dose of drug or medicament from the syringe via the needle (e.g., by way of an injection). The force provided by the driver, in addition to providing a force for injection, can be used to provide some other form of action or motion e.g., retraction, insertion, indication, or other ancillary function of the autoinjector. The canister can include a liquefied gas such as n-butane, nitrous oxide (N₂O) or carbon dioxide (CO₂) for delivering a flow of gas at a vapor pressure of P1. Alternative liquefied gases applicable to the present invention can also be used.

Note here that, as used herein, “liquefied gas” is used to refer to a gas that has been compressed to its vapor pressure so that an equilibrium exists within the vessel in which it is contained such that some portion of the volume is liquid. Advantageously, it is known from thermodynamics that materials in their liquid form require much less space than in their gaseous form, often several hundred times less space. The pressure required for common liquefied gases at room temperature range from around 17 psi for n-butane, around 760 psi for nitrous oxide and around 850 psi for carbon dioxide. In addition, combinations of gasses can be used to modify the pressures to around a particular desired pressure. For example, specific hydrocarbon propellants (e.g., butane, isobutane, and propane) can be mixed in varying quantities in a known manner to obtain pressures ranging from over about 17 psi to about 108 psi. Practically any pressure within the n-butane to carbon dioxide range can be obtained by mixing various gases having differing vapor pressures. A liquefied gas stored in a closed container has its internal pressure directly related only to its temperature and, for a fixed temperature, the pressure generally remains effectively constant until all the liquid portion has boiled off into the gaseous state. The use of a liquefied gas at the appropriate pressure in the manner described herein can provide advantages over present autoinjector technology because it allows for construction of an actuator assembly that can operate as a compact energy and constant pressure source. In addition, and advantageously, actuator assemblies can be constructed as described herein using a liquefied gas at a higher pressure than would be needed and regulate the pressure down to the desired use pressure. In doing so, advantages over conventional autoinjectors can be obtained.

The use of the term “compressed gas” as used herein means a gas that is stored at a pressure and temperature where the gas is never liquefied. With compressed gasses, as the gas is expelled from the container in which it is stored, the internal container pressure decreases. Common examples of such containers are SCUBA air tanks, which are commonly pressurized to around 3000 psi and compressed natural gas (CNG) tanks, which are commonly pressurized to about 2900-3600 psi. With compressed gasses, a pressure-regulating device must be used to obtain a constant pressure. In addition, because no liquefying occurs, the use of compressed gas is less desirable than liquefied gas because the container will tend to be larger and, due to the strength needed to contain the higher pressures, may be heavier as well. Compressed gas also loses pressure over time as it is being spent. In contrast, liquefied gas does not lose pressure over time but instead provides a constant pressure source.

Finally, it should be noted that, as used herein, the terms “propellant,” “liquefied gas” or “compressed gas” are intended to also include gasses that may be the result of a chemical reaction within, or associated with, the storage container, in the instant example, the canister. Since the use of a particular “propellant,” “liquefied gas” or “compressed gas” will be implementation specific, as used herein, the term “driver” is intended to generically encompass “propellants,” “liquefied gases” and “compressed gases,” the selection of which will be a function of the particular intended implementation, and not mandated by the approach itself.

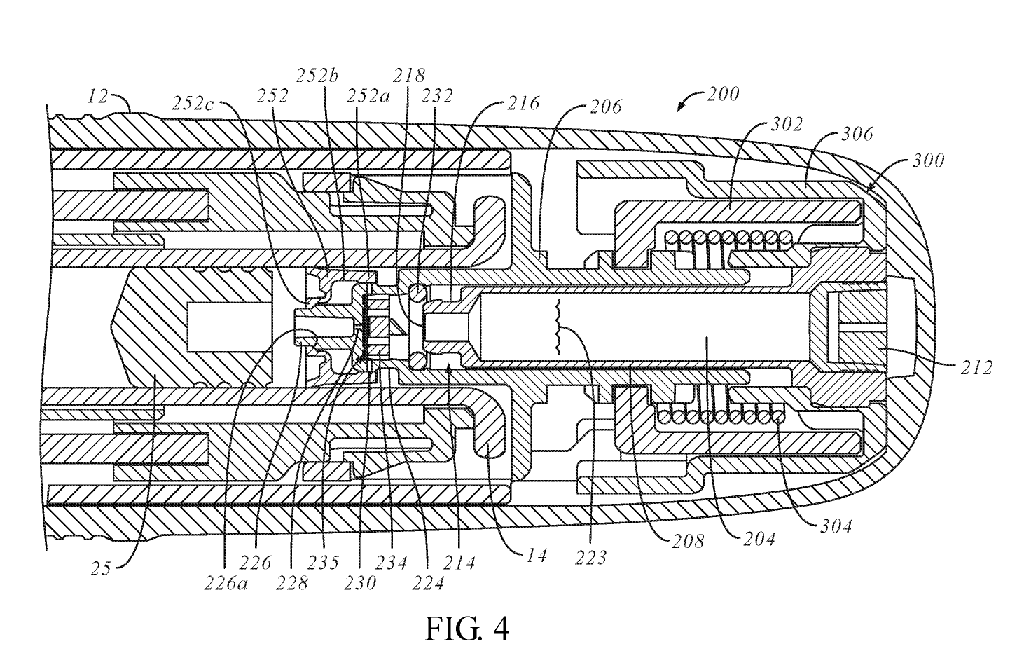

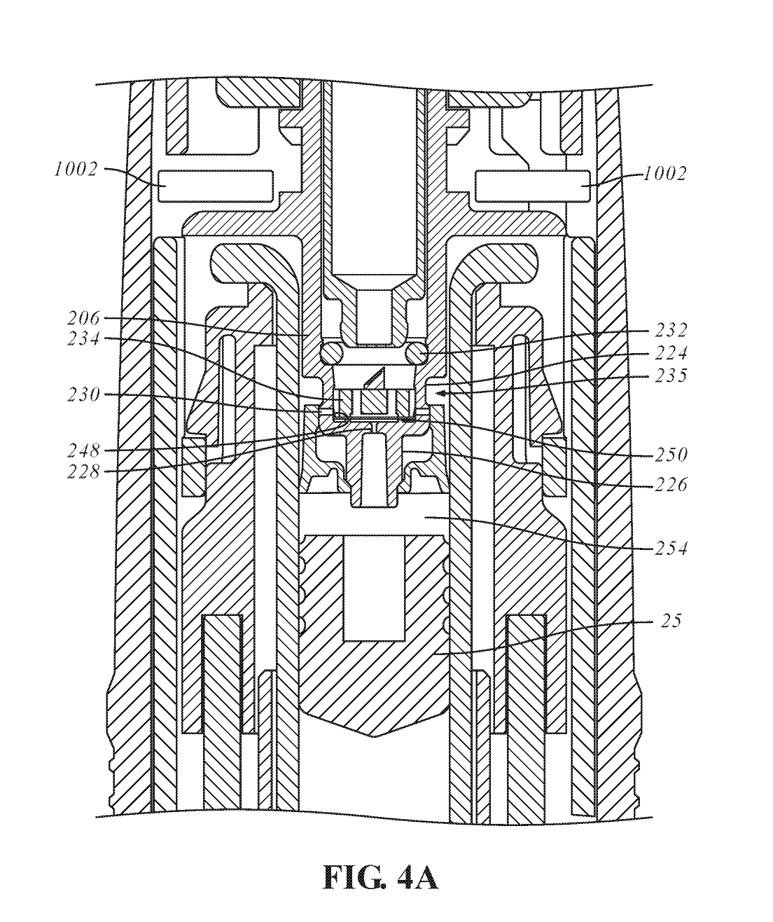

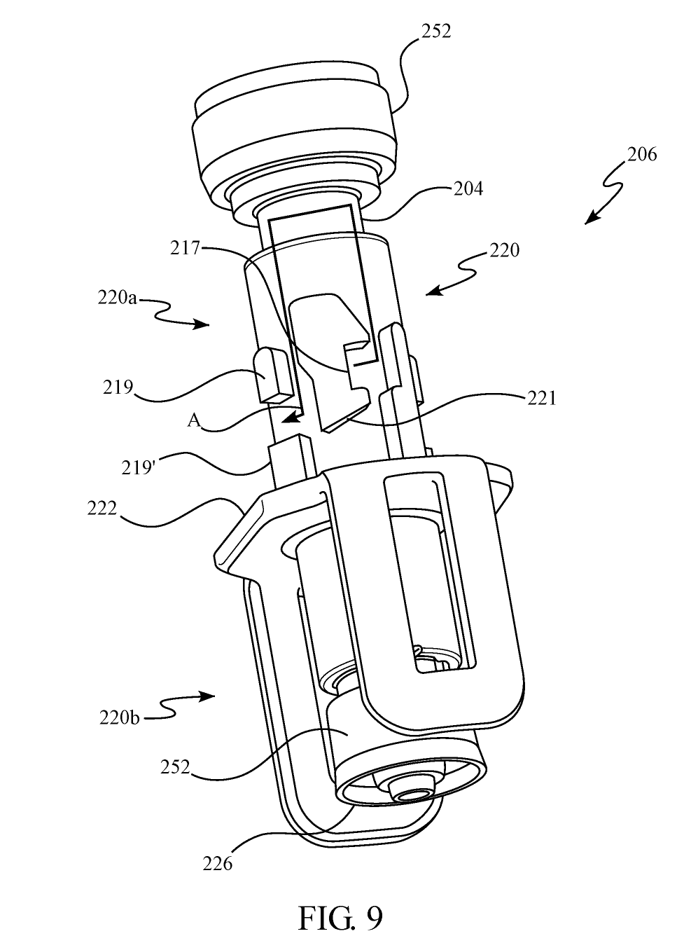

The actuator 206 is configured as best shown in FIGS. 4 and 9, and includes a housing body 220 that includes an upper portion 220a and a bottom portion 220b, a seal 232 and a seal 252. About a midpoint of the actuator is a radially outwardly extending flange 222. Generally everything above the flange 222 is considered the upper portion 220a while everything below and including the flange is considered the bottom portion 220b. About its upper portion 220a, the actuator includes cooperating detents 217, a cam surface 221, and a locking mechanism 219.

The housing body 220 is generally sized and shaped to slidingly receive the canister therein. The actuator is coupled to the canister 204 and a flow regulation device or flow regulator 234. As best shown in FIG. 4A, the actuator 206 is partially housed within the syringe barrel defining a drive chamber or interior volume 254 within the syringe barrel.

The bottom portion 220b of the housing body includes a neck 224 for receiving the distal end of the canister 204, and a nose 226. The nose 226 extends distally from the neck and is configured to have a diameter smaller than the neck.

Centrally located about the distal face of the neck 224 is a through hole or passageway 228 for the passage of gas therethrough and as further discussed below. In other words, the housing body includes the passageway or through hole 228 for receiving a flow of gas provided by the canister downstream of the flow regulation device.

About a distal end of the neck is a substantially radially extending exhaust passageway 230. The neck can include one or more than one, e.g., two or three, exhaust passageways 230.

The seal 232 sealingly engages an inner surface of the actuator and the distal end 214 of the canister. The seal is positioned along a flange portion formed by the walls of the bottom portion of the actuator transitioning from the bottom portion to the nose. Accordingly, the seal 232 is positioned between the canister 204 and the flow regulation device 234, and between the canister and the actuator 206. Preferably the seal is an O-ring seal for providing a hermetic seal between the canister and an inner wall surface of the actuator 206 when the canister is driven distally within the actuator for engaging the seal.

The flow regulation device 234 is configured as best shown in FIGS. 4, 10 and 11 and situated within the distal end 214 of the actuator adjacent the through hole 228. The flow regulation device includes a substantially cylindrical body 236 having a plurality of apertures or grooves 238 extending through the body from its proximal surface 240 to its distal surface 242. Extending outwardly from its proximal surface 240 is a piercing member 244. The piercing member 244 can be, for example, a cannula, a stake, a protuberance and the like which is sufficient to pierce the seal 218 of the canister. Piercing member 244 is designed to engage and pierce the seal 218 of the canister 204 when the canister is driven distally within the actuator 206. The body 236 also includes a chamfer 248 about its distal end.

The flow regulation device 234 also includes a porous member 246 attached to the distal surface 242 for receiving a flow of gas from the canister. The porous member 246 is preferably a porous film having a porosity sufficient to control (e.g., decrease) the rate of flow of gas from the canister towards the through hole 228. The degree of porosity of the porous member 246 is used to control the rate of flow of gas through the flow regulation device.

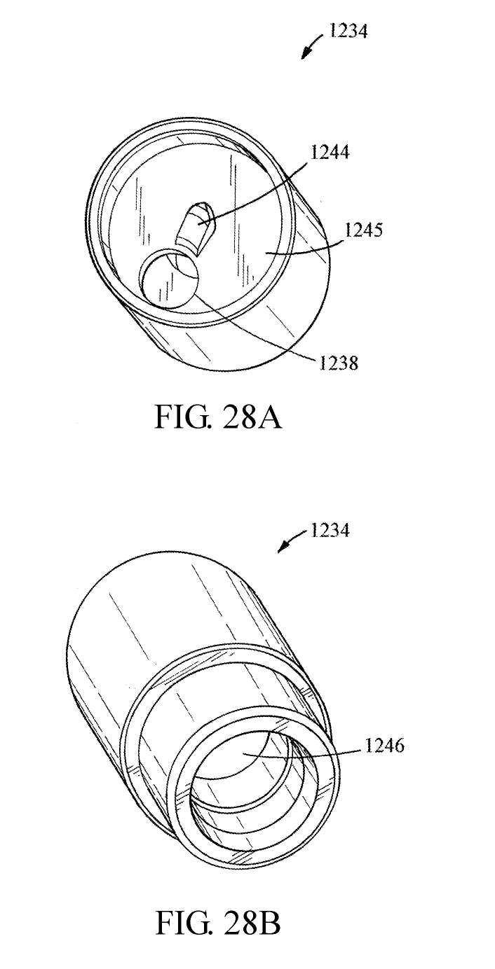

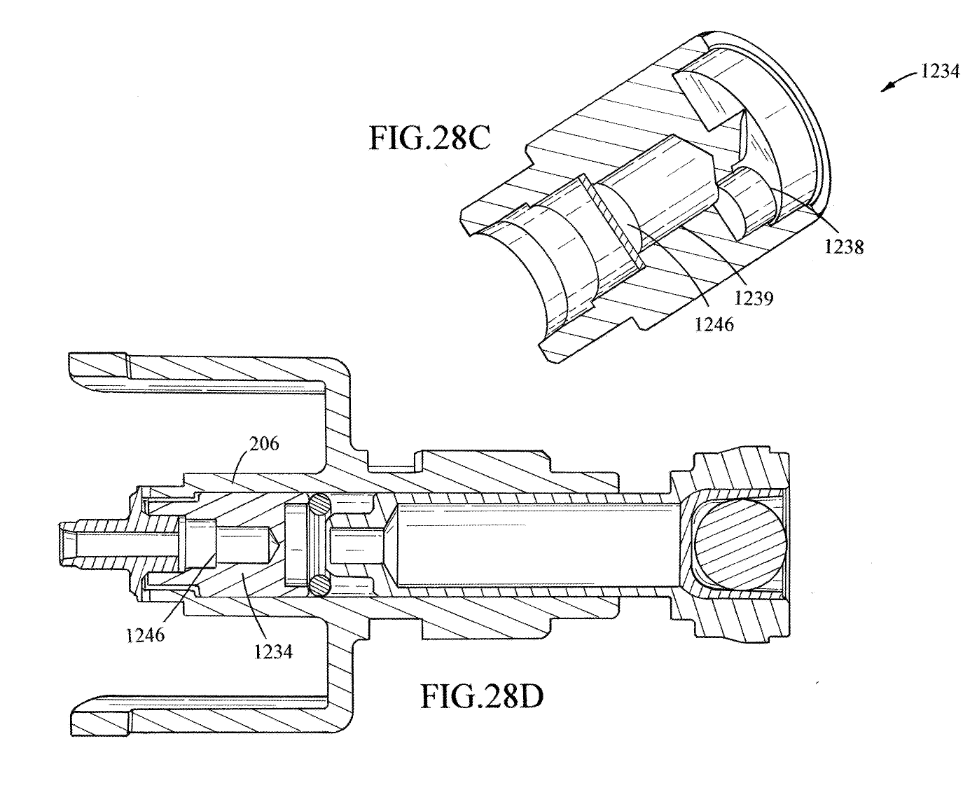

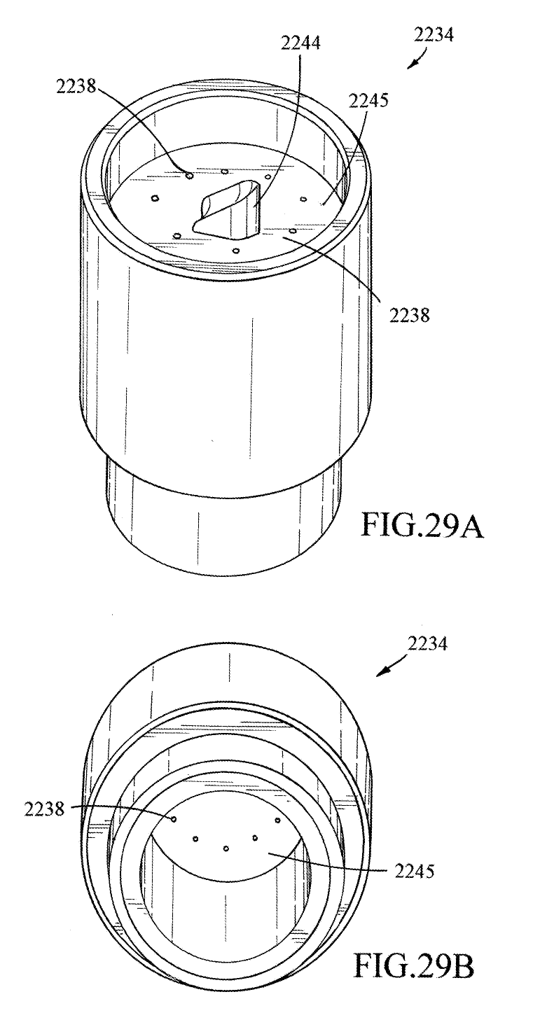

FIGS. 28A-30C illustrate alternative embodiments of a flow regulation device applicable to the present invention. FIGS. 28A-D illustrate flow regulation device 1234 having a piercing member 1244 and an aperture 1238 extending through an upper face 1245 of the flow regulation device. The aperture 1238 is in fluid communication with a plenum 1239 which is in fluid communication with a porous disc 1246. The porous disc 1246 is preferably press-fitted into a bottom counter-bore of the flow regulation device. The flow regulation device can be formed out of a metal or rigid polymer. For example, the porous disc can be a titanium disc having laser drilled holes of a size e.g., about 0.001 inches in diameter. In this embodiment, the flow of gas can be controlled by the apertures 1238. In sum, the flow regulation device includes a body, a piercing member extending from the body for piercing the canister, a passageway (formed by aperture 1238 and plenum 1239) through the body for receiving the flow of gas, and a porous member e.g., porous disc 1246 covering the passageway for controlling the flow of gas through the passageway.

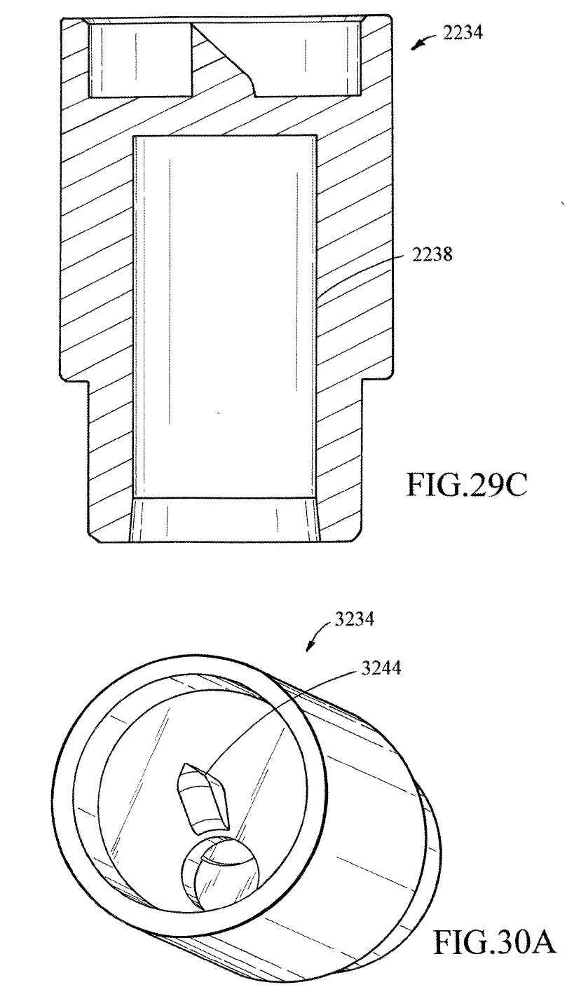

FIGS. 29A-C illustrate flow regulation device 2234 having a piercing member 2244. The flow regulation device has an upper face or proximally facing wall portion 2245 with a plurality of apertures 2238 for controlling and allowing the passage of gas through the upper face. For example, the flow of gas through the flow regulation device 2234 can be controlled by the number and size of the apertures 2238. Similar to flow regulation device 1234, the flow regulation device can be formed out of a metal or rigid plastic. In sum the flow regulation device includes a body having a central through hole 2238, a porous member (e.g., upper face 2245) extending across the through hole about a proximal end of the body, and a piercing member extending from the porous member.

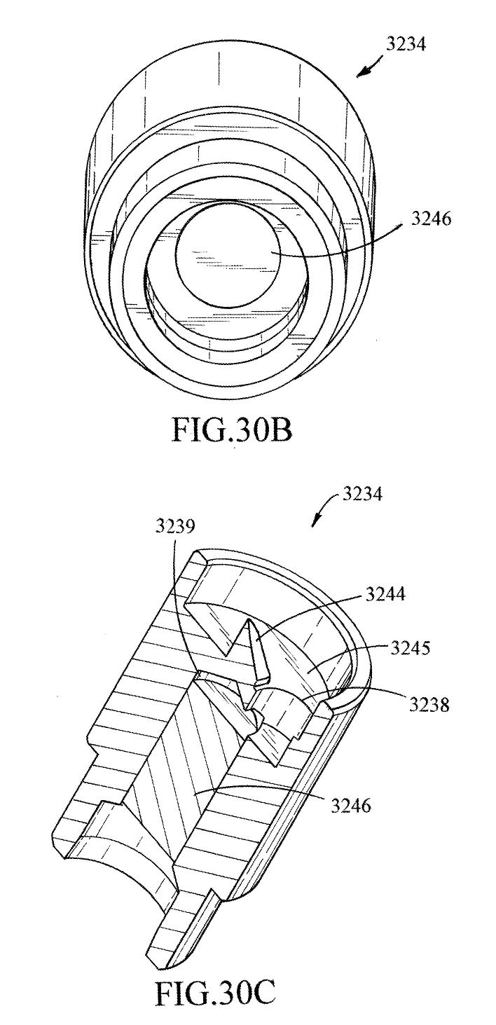

FIGS. 30A-C illustrate flow regulation device 3234 having a piercing member 3244. The main body of the flow regulation device 3234 is configured similarly to flow regulation device 1234, but includes a porous pellet 3246 instead of a porous disc. The porous pellet 3246 can formed out of steel e.g., stainless steel. Preferably, the porous disc is formed out of compressed steel dust. The flow regulation device also includes an aperture 3238 within an upper face 3245 that is in fluid communication with a plenum 3239 within which the porous pellet seats within. In this embodiment, the flow through the flor regulation device can be controlled, in part, through the porosity of the porous pellet. In sum, the flow regulation device includes a body, a piercing member extending from the body for piercing the canister, a passageway (formed by aperture 3238 and plenum 3239) through the body for receiving the flow of gas, and a porous pellet blocking an end of the passageway for controlling the flow of gas through the passageway.

FIG. 4A is an enlarged cross-sectional view of the flow regulation device 234 positioned within the distal end of the actuator 206. Extending inwardly from the distal face of the actuator 206 is a protuberance 250 sized to offset the flow regulation device 234 from a distal facing surface of the actuator. Specifically the protuberance 250 is sized to position the distal surface 242 of the flow regulation device in line with the opening of the exhaust passageway 230. This allows for a flow of gas to the exhaust passageway from the flow regulation device. The protuberance 250 can be configured as a circular protuberance, a plurality of spaced apart protuberances collectively forming a substantially circular structure, a set of three or four spaced apart protuberances or the like.

The protuberance 250 properly spaces the flow regulation device from the through hole 228 thereby allowing for a flow passageway in a direction from the through hole to the exhaust passageway 230 upon a completion of an injection by the autoinjector, as further discussed below. Additionally, the chamfer 248 on the flow regulation device facilitates the free flow of gas through the exhaust passageway by eliminating surfaces on the body 236 that could potentially block the opening of the exhaust passageway 230.

The combination of features about the distal end of the actuator 206 is collectively referred to as a hub assembly 235. The hub assembly includes the flow regulator 234 and protuberances 250. As such, the actuator includes a hub assembly having a piercing member and the hub assembly engages the canister in the engaged position and is spaced from the canister in the disengaged position (FIG. 4A).

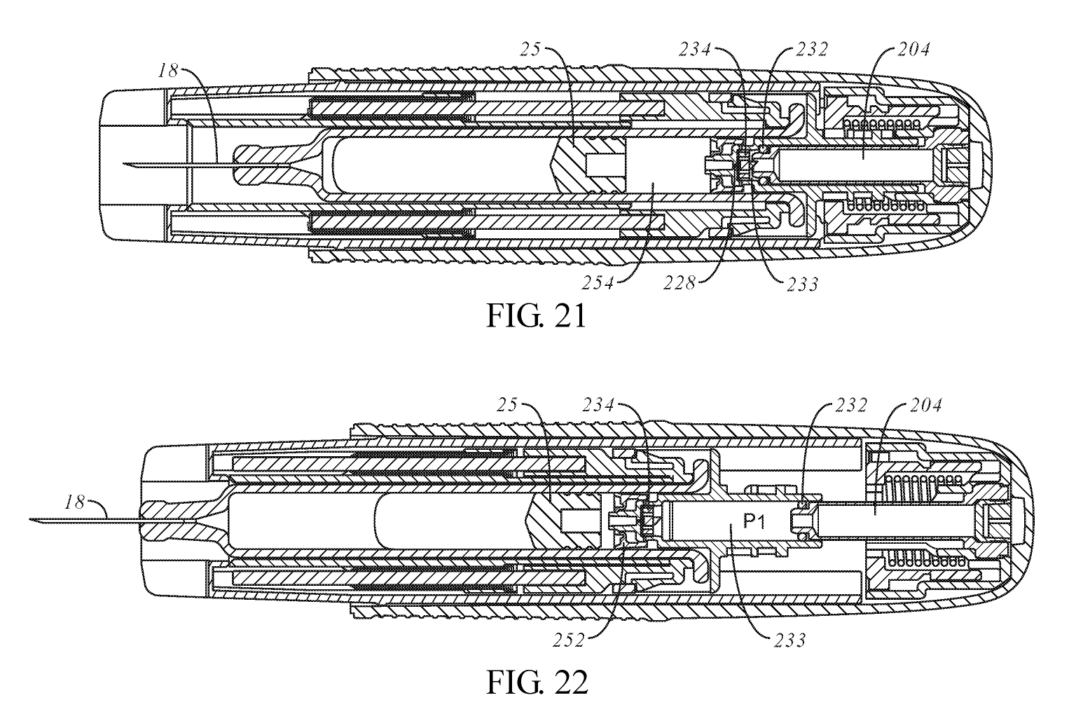

FIG. 4 illustrates the actuator 206 in a storage or initial position and FIG. 21 illustrates the actuator 206 in an engaged position or activated state. That is, the actuator 206 moves relative to the canister 204 between the engaged position and the storage position. In the storage position, the flow regulation device 234 is spaced from the canister 204. In other words, the canister is disengaged with the actuator 206 and the through hole 228 is not in fluid communication with the canister or a flow of gas provided by the canister.

In the engaged position, the actuator moves relative to the canister such that the flow regulation device pierces the seal 218 of the canister and the seal 232 sealingly engages the distal end 214 of the canister. Further, in the engaged position the flow regulation device 234 is in fluid communication with the canister for receiving a flow of gas from the canister. Furthermore, in the engaged position, the through hole 228 is in fluid communication with the canister such that the flow of gas from the canister flows through the through hole 228. Alternatively expressed, the canister engages the actuator to allow fluid communication of the flow of gas with the through hole 228.

The actuator seal 252 is configured as best shown in FIGS. 4 and 9. The seal 252 includes a distal end that engages the nose 226 of the actuator and an inner surface of the syringe barrel 20. The proximal end of the seal 252 includes a sealing portion 252a for circumscribing the distal end of the actuator to sealingly engage the exhaust passageway 230 when the actuator is in both the engaged and storage positions as shown in FIGS. 4 and 21. The seal 252 seals off the exhaust passageway 230 such that the flow of gas exiting the canister and flowing through the flow regulation device 234 does not exit the actuator therethrough. Adjacent the sealing portion 252a is a relief portion 252b having an inner diameter greater than an inner diameter of the sealing portion. As such, when the relief portion 252b is adjacent the exhaust passageway 230, the relief portion 252b does not sealingly engage the exhaust passageway and allows for fluid communication through the exhaust passageway 230.

FIGS. 3 and 21 illustrate operation of the autoinjector 10 as the actuator 206 moves from the storage position (FIG. 3) to the engaged position (FIGS. 21). FIGS. 3 and 21-25 illustrate operation of the autoinjector as it moves through all phases of operation. The actuator 206 is activated and driven to the engaged position by the activator assembly 300, as further described below. Once moved to the engaged position, the canister engages the seal 232 defining an actuator plenum 233 between the distal end of the actuator 206, and the canister and seal 232 engagement. Additionally, the flow regulation device pierces the seal 218 of the canister thereby allowing a flow of gas generated by the liquefied gas 223 to flow out of the canister, into the actuator plenum 233 and through the flow regulation device.

The rate of flow through the flow regulation device is controlled by the porous member 246 of the flow regulation device. The flow of gas is directed through the through hole 228 owing to the hermetically sealed interface between the actuator and the canister by the seal 232 and closure of the exhaust passageway 230 by the seal 252. The flow of gas exiting through the through hole 228 of the actuator enters a space within syringe barrel 20 bounded between the plunger 25 and the seal 252. The space bounded between the plunger and seal define an interior volume or a drive chamber 254.

Owing to the sealing engagement of the plunger 25, seal 252, and seal 232 as the flow of gas enters the drive chamber 254 and plenum 233, the pressure within the drive chamber and actuator plenum 233 increases thereby first moving the syringe distally relative to the housing to initiate an insertion of the syringe needle into a patient and then driving the plunger 25 distally within the syringe barrel to effectuate a dispense of the medicament within the syringe.

This sequence of movements of the syringe 14 and the plunger 25 is accomplished by balancing the pressures within the actuator and syringe barrel against the compressive forces of the retraction biasing members 42, 44 and the stiction forces of plunger 25 and seal 252. Specifically, the syringe via the cradle is allowed to move distally to effectuate an insertion of the needle upon the pressure within the actuator plenum 233 reaching a first predetermined pressure P1 (FIG. 22). In other words, due to the predefined stiction forces between the plunger 25 and seal 232, the plenum 233 expands before the piston travels distally with in the syringe barrel.

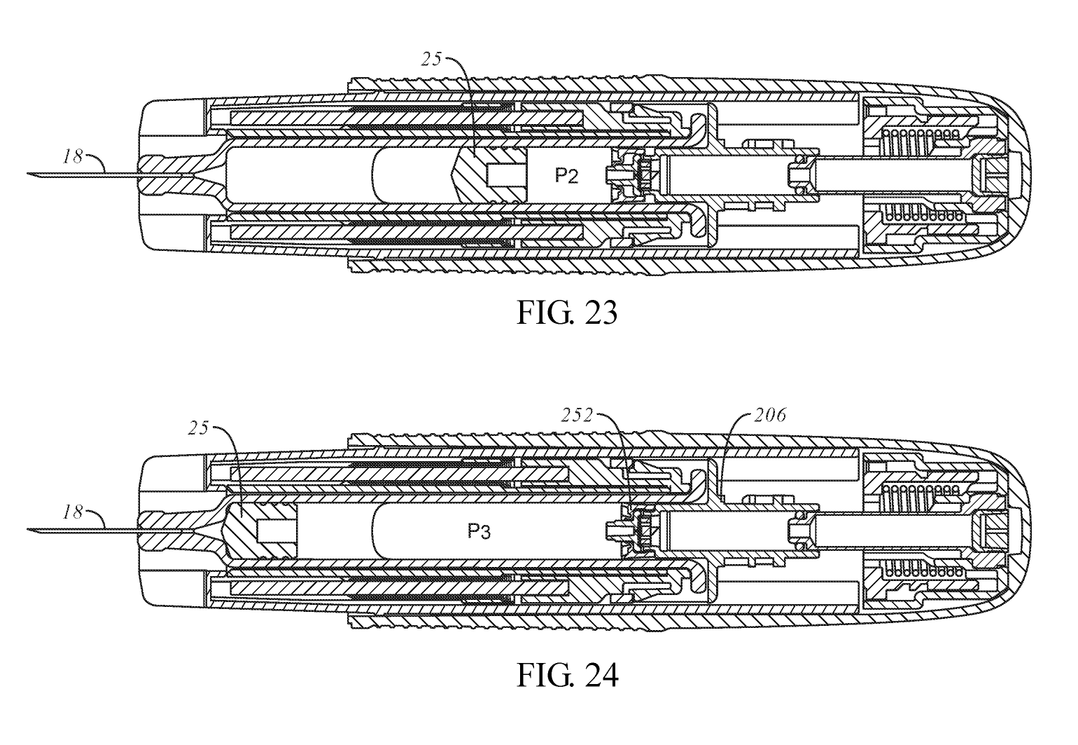

Then upon the pressure within the drive chamber 254 exceeding a second predetermined pressure P2, which is greater than the first predetermined pressure P1, the plunger 25 is driven distally within the syringe barrel (FIG. 23). This movement is accomplished in part as a result of the plunger 25 having a stiction force less than a stiction force of the seal 252, and greater than the force necessary to expand the retraction biasing members. The second predetermined pressure P2 is less than a required pressure necessary to drive movement of the seal 252 i.e., a pressure less than that required to exceed the stiction forces holding the seal 252 in the initial position.

Consequently, as the plunger 25 moves distally down the length of the syringe barrel, the volume of the drive chamber 254 increases as the flow of gas continuously enters thereby substantially maintaining the pressure within the drive chamber at pressure P2 until the plunger 25 bottoms out at the distal end of the syringe barrel. Upon the plunger reaching the end of its stroke length i.e., reaching the distal end of the syringe barrel, the volume of the drive chamber remains fixed and the pressure therein increases as result of the continuous flow of gas entering the drive chamber. Then as more gas enters the drive chamber, the pressure therein increases to a third predetermined pressure P3 which is sufficient to overcome the stiction forces of the seal 252 and the mechanical interference between an inner ring 252c of the seal 252 and a lip 226a of the nose 226 (see FIG. 4) which forms a larger diameter nose portion. As a result, the seal 252 is driven in the proximal direction towards the actuator 206 and moves the seal 252 from a first position where the sealing portion 252c sealingly engages the exhaust passageway 230 towards a second position where the relief portion 252b is adjacent and spaced from the exhaust passageway 230 (FIG. 24).

The movement of the seal from the first position to the second position is controlled by the configuration of the seal and nose interaction. That is, configuration of the seal's inner ring 252c and the nose lip 226a establishes a predetermined force necessary to overcome to move the seal from the first position to the second position. Alternatively, this mechanical interaction for establishing the predetermined force can be established by alternative mechanisms, e.g., a wire ring surrounding the nose and the like.

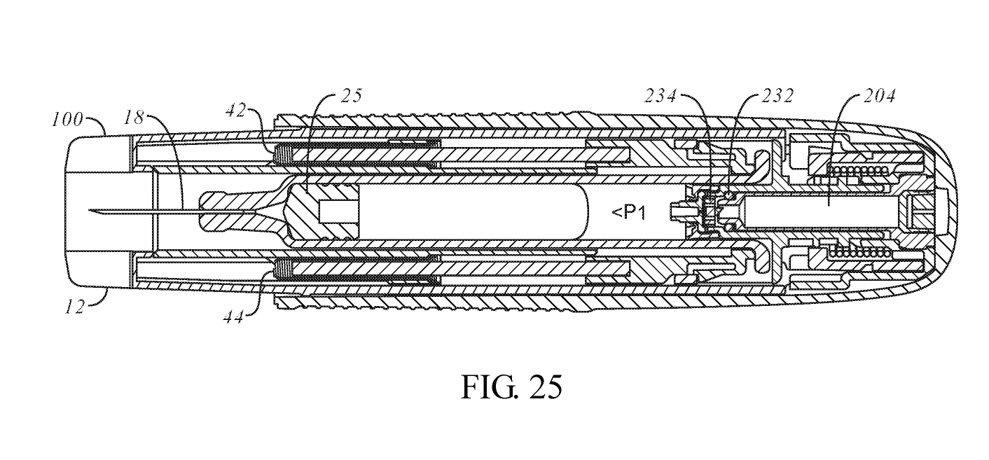

Upon movement of the seal 252 from the first position to the second position, the flow of gas continues to exit from the canister, through the flow regulation device, and out through the exhaust passageway 230, thereby allowing the autoinjector to vent its remaining liquefied gas. After the exhaust passageway 230, the gas exits the autoinjector through a series of spacings between the various components of the autoinjector. In other words, the pressure P3 within the drive chamber of syringe barrel travels back up through the through hole 228 and out the exhaust passageway 230 and then ultimately exiting the autoinjector thereby relieving the pressure within the drive chamber to a pressure less than pressure P1 such that the retraction biasing members 42, 44 can bias the syringe in a proximal direction relative to the housing to retract the syringe needle within the confines of the shield 100 (FIG. 25).

Referring to FIG. 4A, the autoinjector can optionally include a secondary chamber 1002 that is in fluid communication with the exhaust passageway 230 such that excess gas gets expelled and trapped within the secondary chamber. In this embodiment, excess gas from the autoinjector's canister and drive chamber is retained within the autoinjector itself and prevented from being vented to atmosphere.

Referring back to FIG. 25, the flow of gas existing the canister and vented to an exterior of the housing travels along a defined passageway or venting passageway. For example, the passageway can extend from the through hole 228 to an exterior of the housing. The passageway also includes the exhaust passageway 230 about a distal end of the actuator. Further the passageway includes a spacing between the seal 252 positioned within the syringe barrel and the actuator. Specifically, the seal 252 is moveable between a first position sealingly engaging the exhaust passageway, and a second position spaced from the exhaust passageway. When the seal is spaced from the exhaust passageway, it defines the spacing between the seal and the actuator of the defined passageway.

The collective effect of how the pressures are supplied by the liquefied gas within the autoinjector provide an adaptive force for applying to the piston to drive the medicament from the medicine cartridge chamber. The adaptive force increases or decreases based upon a change in speed of travel of the piston. For example, as a flow of gas provided by the liquefied gas exits the canister, it applies an adaptive force to the piston in a controlled manner such that: i) the adaptive force will be constant at a constant injection rate, ii) the adaptive force will increase, if the injection rate slows to below the constant injection rate, and iii) the adaptive force will decrease, as the injection rate increases from below the constant injection rate towards the constant injection rate. The adaptive force provided by the liquefied gas within the autoinjector can be as described in U.S. Patent Application Publication Nos. 2014/0114248 and 2014/0114250, the entire disclosures of which are hereby incorporated by reference herein for all purposes.

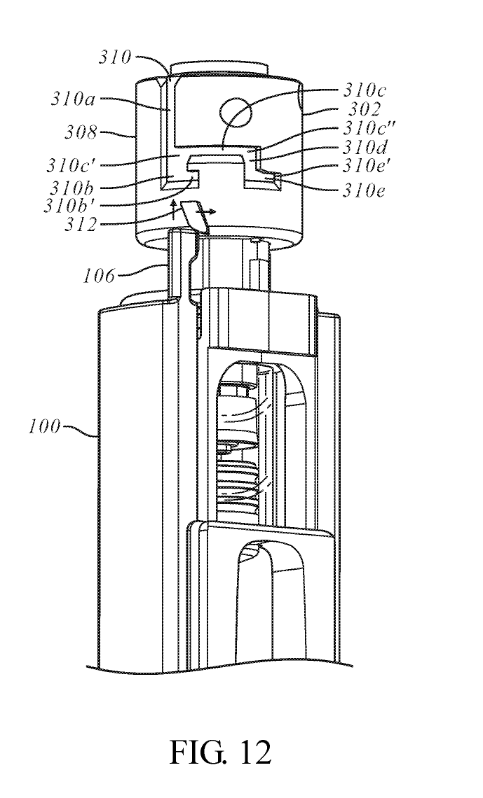

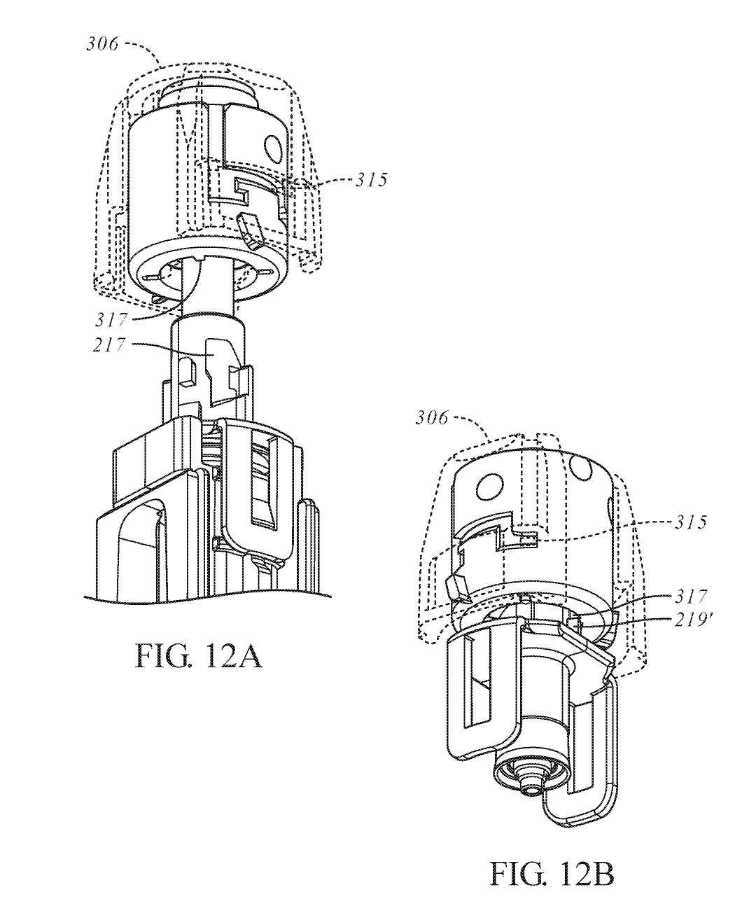

Referring to FIGS. 12-19 the activator assembly 300 includes an activator 302, a biasing member 304, and a cap or activator housing 306 having a tab 315. The activator 302 is situated about a proximal end of the housing 12 and receives the actuator 206. The biasing member 304 is positioned within the activator and the cap 306 covers the activator.

The activator 302 is configured as best shown in FIGS. 12-14. The activator has a substantially tubular body 308, a track 310, and a cam 312 having a cam surface.

The activator circumscribes the actuator 206 and is moveable relative to the actuator. In one aspect, the activator is rotatable about the actuator. The activator is moveable relative to the actuator between an initial position, an activated position, and a retracted position. In the initial position, a distal end of the actuator is spaced from a distal end of the activator at a first distance (FIG. 12). In the activated position (FIG. 12A), the distal end of the actuator is spaced from the distal end of the activator at a second distance. In the retracted position (FIG. 12B), the distal end of the actuator is spaced from the distal end of the activator at a third distance. The actuator is also locked in the retracted position by the activator, as further discussed below. Preferably, the first distance differs from the second distance, and the second distance differs from the third distance.

The biasing member 304 also biases the activator and the actuator relative to each other such that the biasing member biases one of the actuator and activator to move between the initial position and the activated position upon engagement of the cam surface. As further discussed below, the cam surface is engaged upon by the leg 106 of the shield.

The track 310 is formed about and preferably within the side wall of the tubular body. Preferably, the activator includes two tracks 310 and 310′. The track 310′ is configured similar to track 310. The track includes a vertical extent or axial track portion 310a, a first horizontal extent or circumferential track portion 310b, a second horizontal extent 310c, a second vertical extent 310d, and a third horizontal extent 310e. The first vertical extent 310a is in fluid communication with the first horizontal extent 310b, and each are in fluid communication with the second horizontal extent 310c. The second horizontal extent 310c is spaced from the first horizontal extent 310b. The second vertical extent 310d is in fluid communication with second horizontal extent 310c and the third horizontal extent 310c. The track 310 is configured to engage and/or receive the corresponding tab 315 on the cover 306, as further discussed below.

One end of the first horizontal extent or first end 310b defines a first position 310b′ or initial position for engagement with the tab 315. As shown in FIG. 12, about a left most end of the second horizontal extent 310c defines another position 310c′ and a right most end or second end of the second horizontal extent 310c defines a second position 310c″ for the tab. The second position 310c′ is circumferentially spaced from the first position 301b′ in a first direction for engaging the tab when the activator is beginning its activation phase. The third position 310c′ is circumferentially spaced from the second position in a second direction opposite the first direction, and circumferentially spaced and axially spaced from the first position. The third position 310c″ corresponds to the autoinjector's needle injection phase. The third horizontal extent 310e defines a fourth position 310e′ that is circumferentially spaced from the first, second, and third positions, and axially spaced from the third position. Accordingly, the activator is moveable relative to the actuator to the fourth position circumferentially spaced from the third position. The fourth position corresponds to the autoinjector's retracted position.

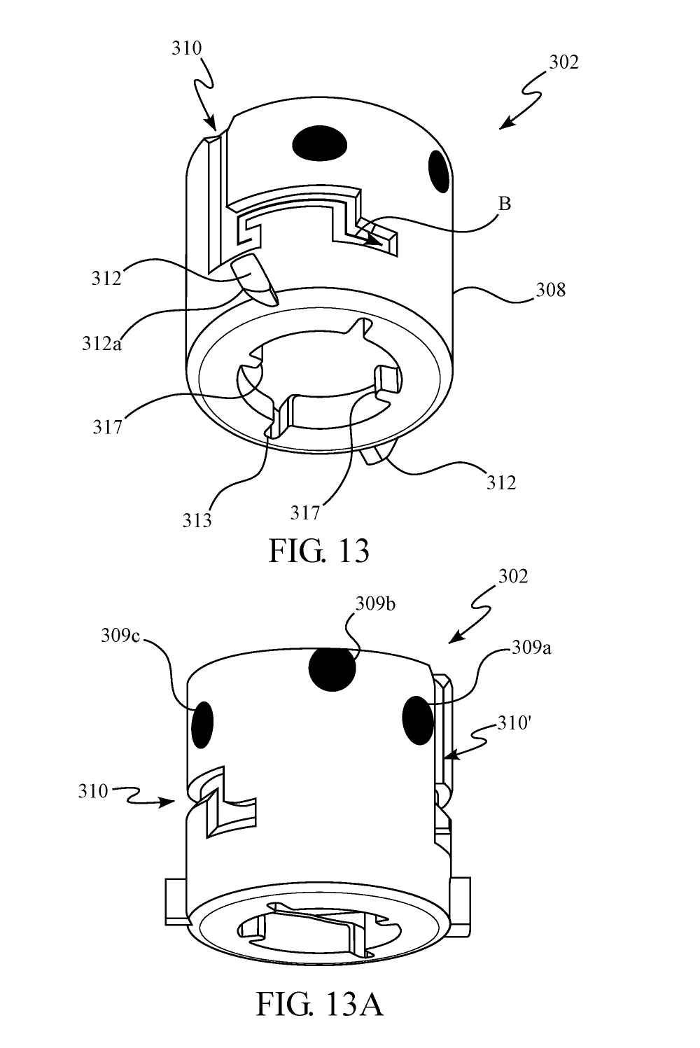

The cam 312 is configured as best shown in FIGS. 13 and 14 and positioned about a bottom end or distal end of the activator 302. Preferably, the cam is positioned spaced from the track 310. The cam 312 includes a camming surface or cam surface 312a. Specifically, the camming surface is a non-vertical, non-horizontal camming surface. The cam is configured to engage the leg 106 of the shield 100. Preferably, the activator includes a pair of cams, for respectively engaging each of the legs 106 of this shield 100. Owing to the angle of the camming surface 312a, the activator 302 rotates towards the right when viewed as shown in FIG. 12.

In sum, the activator 302 is coupled to the actuator and moveable relative to the actuator between the first position, the second position, the third position and the fourth position. In the first position, the actuator is stationary with respect to the activator. In the second position, the activator has been rotated relative to the actuator so as to be circumferentially spaced from the first position by interaction of the leg 106 with the com 312. In the third position, the activator has been further rotated relative to the actuator so as to be circumferentially spaced from the second position. The biasing member 304 biases the activator and actuator relative to each other to move one of the activator and actuator between first, second and third positions. In the present embodiment, the activator moves relative to a stationary actuator. Then owing to the forces supplied by the biasing member 42, 44 and 304, the activator is subsequently moved to the fourth position 310e′.

Referring to FIGS. 13, 14 and 18, the activator includes a slot 313 for receiving and/or engaging the biasing member 304, and a tab or detent 317 for engaging a cooperating detent 217 in an initial position or a locking feature or stop 219 in a subsequent retracted position on the actuator 206. That is, the activator detent 317 moves along a path illustrated by arrow A (FIG. 9). The path travelled by detent 317 corresponds to the path traveled by tab 315 along the track illustrated by arrow B in FIG. 13. Thus, when the tab reaches the fourth position along the track i.e., the third horizontal extent 310e, the detent 317 is situated within or adjacent locking feature 219, 219′ (FIG. 12B). In other words, the biasing members 42, 44, 304 biases one of the actuator and activator to move to a final position whereby the activator engages the locking feature 219 of the actuator. In sum, the actuator includes a cam surface and the stop 219, and the activator includes detents 317 that engages detents 217 of the actuator in the first position and engages the cam surface 221 in moving from the first position to the second position.

Referring to FIG. 13A, the activator also includes a series of indicators 309a, 309b and 309c that each correspond to a particular state of operation of the autoinjector and is viewable by the user through respective windows 47a, 48b and 48c (FIGS. 26A, 26B, 26C) of the housing. Each indicator is configured as a visually identifiable feature positioned about the activator so as to be viewable by a user. The indicators can be of the same or of varying colors so as to be color coded to their respective indication of the autoinjector state. Preferably, each indicator is configured as a color patch, but can alternative be configured as any other visually identifiable feature suitable for its intended purpose e.g., a light emitting diode (LED) and the like.

Indicator 309a is positioned on the activator such that it is visible through window 48a when the autoinjector is in the initial or ready to use state and the cap is in the first position on the activator (FIG. 26A). Indicator 309b is positioned on the activator such that it is visible through the window 48b when the autoinjector is in the activated insertion or injecting state and the cap is in the third position on the activator (FIG. 26B). Indicator 309c is positioned on the activator such that it is visible through window 48c when the autoinjector is in the retracted state and the cap is in the fourth position on the activator (FIG. 26C).

Alternatively, the various indicators can be used to indicate various other states of the autoinjector as it moves through its injection process. For example, indicator 309b can be used to indicate an initial or ready to use state, or a retracted state. Likewise, indicators 309a and 309c can be used to indicate other states of the autoinjector. Further, alternative forms of indicators besides those shown in FIGS. 26A-C can be used, e.g., color coded indictors or symbols or markings can be used.

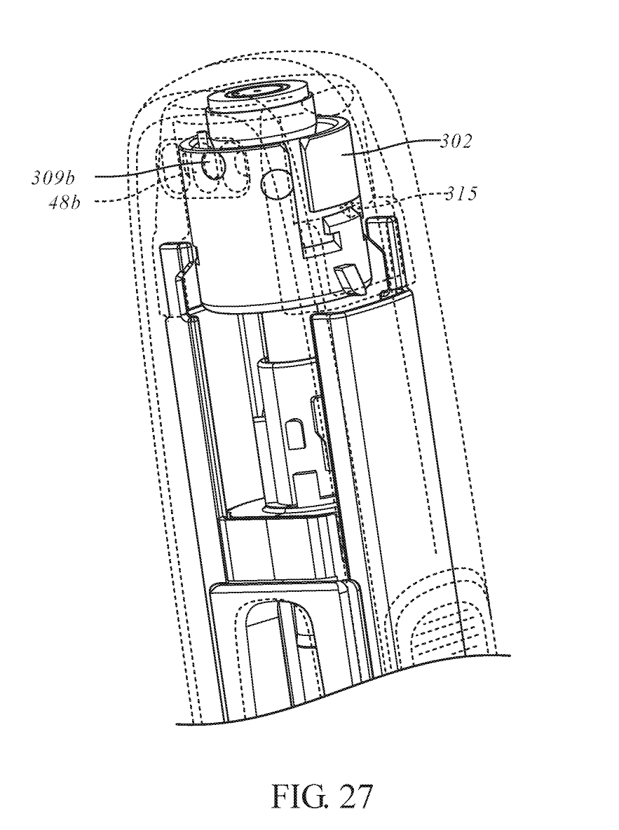

FIG. 27 illustrates the positioning of indicator 309b viewable through window 48b while the cap's tab 315 is in the third position along the activator's track 310.

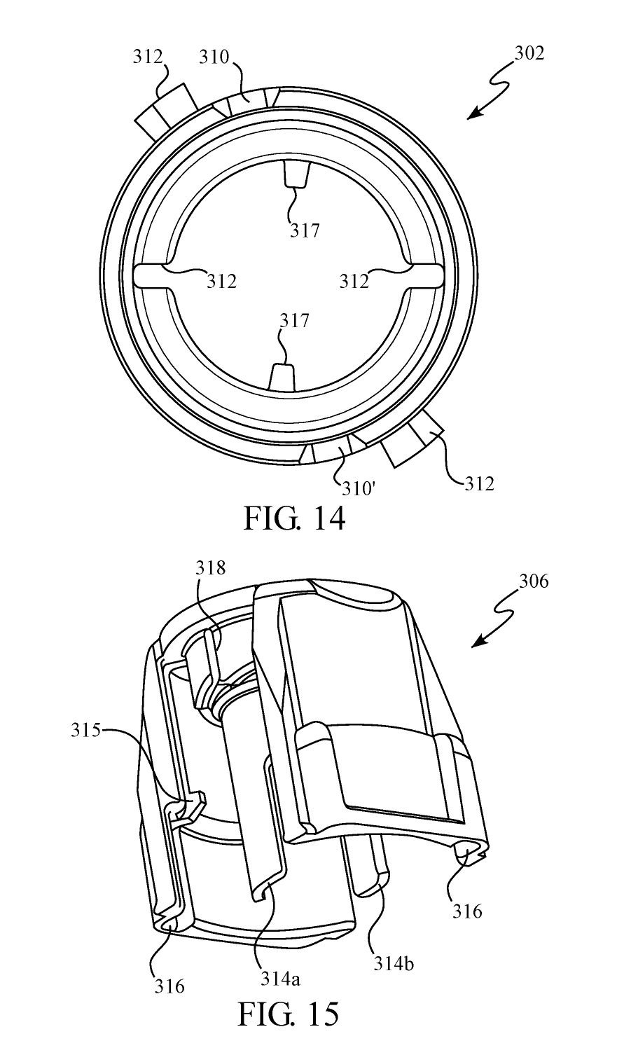

The cap 306 is configured as best shown in FIGS. 15-17. The cap 306 includes an inner pair of arms 314a, 314b radially spaced from the side walls of the cap. The arms 314a, 31b are sized and configured to receive the actuator 206, as best shown in FIG. 2. The tab 315 extends inwardly from the side wall of the cap and positioned about the cap so as to be slidingly received within the track 310 when the cap is assembled to the activator 302. In accordance with an aspect of the present embodiment, the tab directly engages the track to releasably maintain the position of the actuator relative to the activator in the initial position (FIG. 12), activated position (FIG. 12A) or retracted position (FIG. 12B).

The cap also includes a track 316 formed about or within a side wall for receiving and housing the leg 106 of the shield 100. Preferably, the cap includes a track formed within each side wall so as to receive and house each of the pair of legs 106 of the shield. Further, the cap includes a slot 318 for receiving and/or engaging the biasing member 304.

Referring back to FIGS. 2, 3, 9 and 16, the canister 204 is mounted to the cap. Specifically, the canister engages an inwardly extending flange 319 of the cap so as to seat and be housed within a recess 321 of the cap.

Referring to FIGS. 4 and 19, the biasing member 304 is preferably configured as a coil spring having a first end engaged with the slot 318 of the cap and a second end engaged with the slot 313 of the activator. Alternatively, the biasing member can be configured as any other biasing member suitable for biasing the activator relative to the cap, such as, an elastomeric member, a leaf spring, and the like. The biasing member provides a rotational biasing force and an axial biasing force to the activator and actuator.

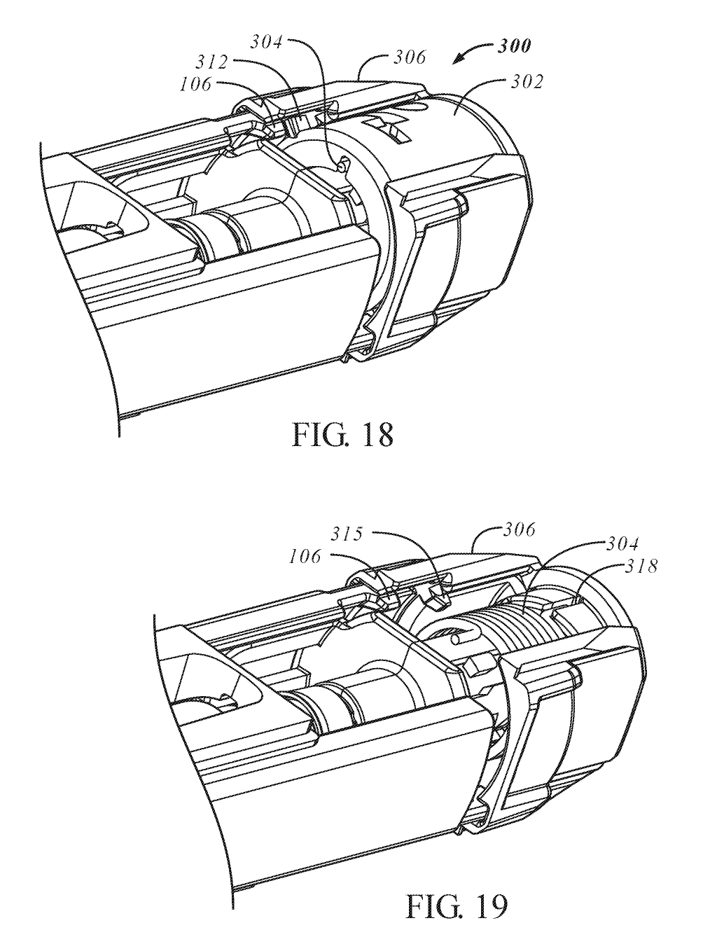

FIGS. 18 and 19 illustrate the cap 306 assembled to the activator 302, the biasing member 304 and the shield 100 of the autoinjector. The leg 106 is received within the track 316 of the cap and slightly spaced from or engaged with the cam 312. The tab 315 is situated within the first position of the track 310.

Upon actuation of the autoinjector, the shield is forced to move distally relative to the housing thereby causing the legs 106 to cam against the cam 312. The camming action between the legs and the cams cause the activator to rotate in a first direction relative to the cap. In doing so, the cooperating detents 317 and 217 disengage thereby allowing the biasing member 304 to move the activator distally relative to the actuator causing the canister to move and be punctured by the flow regulator to allow pressure buildup within the plenum 233 to move the cradle 34 relative to the housing (FIG. 12A). The tab 315 is also caused by basing member 304 to move upwards from the first position towards the second position adjacent the horizontal extent 310c of the track, which upon doing so causes the tab to enter the second horizontal extent owing to the biasing force of the biasing member 304 biasing the activator in the second direction opposite the first direction. As a result, the tab 315 moves towards the third position, and then to the fourth position, as a result of the forces applied by the biasing members 304 and 42, 44.

Operation of the autoinjector can be broken down into several operational states, namely an initial state (FIG. 1), an armed or ready to use state or first position (FIGS. 2, 3 and 20), an activated state (FIG. 21), needle insertion state or second and third positions (FIG. 22), injecting state (FIG. 23), an end of dose state (FIG. 24) and a retracted state or fourth position (FIG. 25). Starting from the initial state a user arms the autoinjector by grasping the distal end or second section 26 and separating it from the autoinjector or first section 24. Now the autoinjector is in the ready to use state as shown in FIG. 20.

To activate the autoinjector, or in other words to administer a self-injection, the user grasps the autoinjector, preferably grasping the first section 24, and pressing the distal end against the injection site. This pressing action results in the housing, and consequently the cradle 34, to move relative to the shield 100. Specifically, the housing and cradle moves towards the shield. Further, owing to the design of the autoinjector 10, once the shield is pressed against an injection site, the injection phase of the autoinjector begins without any primary or residual forces acting on the shield 100 to urge the shield away from the injection site. In other words, the autoinjector does not include any biasing member or forces acting on the shield to move the shield towards the housing after the injection phase begins, nor does the shield move or travel relative to the housing after the injection phase has begun.

As a result, the legs 106 at the proximal ends of the shield are caused to engage the cams 312 of the activator to disengage the cooperating detents 317 from the actuator 206, thereby allowing the actuator to separate or move from the activator 302. That is, when moving from the initial position to the injection position the proximally extending latch member engages the activator cam surface 312 to move the activator from the first position to the second position along the track 310. Concurrently, the actuator 206 is moved relative to activator 302 and consequently the canister 204. As such, the canister engages the seal 232 and the flow regulator 234 pierces the pierceable seal 218. This results in the liquefied gas at a pressure of P1 to flow through the flow regulator and into the drive chamber 254 and plenum 233. In other words, the flow regulator engages the canister to receive a flow of gas generated by the liquefied gas and the flow of gas enters the drive chamber at a pressure that is less than P1 owing to the larger volume of the drive chamber.

Once the liquefied gas enters the drive chamber and plenum, the needle insertion state or phase begins. Specifically, the liquefied gas builds up pressure within the plenum sufficiently to cause the seal 232 to overcome applicable stiction forces against the internal walls of the actuator and the biasing force on the syringe provided by the biasing members 42, 44. This buildup of pressure and drive of the syringe is accomplished within a very short time to provide a quick needle insertion into the user.

After the needle insertion is completed, the autoinjector enters the injection state or phase. The liquefied gas continues to build up pressure within the drive chamber 254 sufficiently to overcome the stiction forces on the plunger 25 to drive the plunger distally within the syringe barrel thereby causing the medicament stored therein to be injected out of the syringe needle. Specifically, the pressure within the drive chamber rises to P2 which is less than P1 to drive the piston or plunger distally along the syringe barrel. The pressure within the syringe barrel is substantially maintained at P2 for driving the piston to be fully seated against the distal end of the syringe barrel.

After the injection phase is completed, the liquefied gas continues to build up pressure within the drive chamber 254 sufficiently to overcome stiction forces of the seal 252 to move this seal from its initial position or first position wherein the exhaust passageway 230 is sealed to a second position relative to the actuator whereby the seal 252 is spaced from the exhaust passageway 230. When the seal 252 is moved to the second position, the liquefied gas is allowed to escape out through the exhaust passageway 230 of the autoinjector and subsequently through various passageways and openings of the autoinjector components. In other words, after the piston is fully seated against the distal end of the syringe barrel, the pressure within the syringe barrel increases to P3, which is greater than P2 and less than P1, until the pressure within the syringe barrel moves the seal from the first position to the second position. Owing to the control of pressures within the autoinjector, the piston is driven distally along the syringe barrel at a substantially constant speed by the pressure P2. However, due to the expanding volume of the drive chamber as a result of the moving piston, when the speed of travel of the piston decreases, the pressure within the syringe barrel increases from P2 to P2′, wherein P2′ is greater than P2.

Once all the medicament within the syringe is dispensed and the piston bottoms out within the syringe, the drive chamber volume becomes fixed. As a result, the continued flow of gas entering the drive chamber from the canister increases the pressure within the drive chamber until a pressure P3 is attained. P3 is greater than P2 but less than P1. A pressure of P3 is also sufficient to overcome the stiction forces of the seal 252 pressing against the actuator and syringe barrel. As a result, the seal 252 is driven in the proximal direction towards the actuator 206 and moves the seal 252 from its first position where the sealing portion 252a sealingly engages the exhaust passageway 230 to its second position where the relief portion 252b is adjacent and spaced from the exhaust passageway 230. This then allows for the pressures within the drive chamber and plenum to exit the exhaust passageways and ultimately out of the autoinjector completely.

Once the buildup of liquefied gas within the autoinjector has escaped, the pressure within the drive chamber 254 and plenum 233 drops sufficiently to allow the biasing members 42, 44 to bias the cradle 34 proximally relative to the shield 100 thereby automatically retracting the syringe needle within the shield. Specifically, the biasing members bias the distal body 36a such that the distal body of the cradle 34 moves in the proximal direction relative to the proximal body 36b thereby moving the syringe within the shield 100.

In other words, the cradle assembly is coupled to the medicine cartridge and slidably attached to the shield such that the cradle assembly is moveable between a primary position (corresponding to an initial position), a secondary position (corresponding to an activated position or any position thereafter except a retracted position), and a tertiary position (corresponding to a retracted position) relative to the shield. Cooperating catches on the distal body of the cradle assembly and the shield engages one another to hold the distal body in the secondary position. Specifically, the distal body and proximal body are moveable from the primary position to the secondary position, and the proximal body is moveable from the secondary position to the tertiary position relative to the distal body.

Biasing members coupled to the cradle assembly and shield biases the cradle assembly after venting of the canister. Specifically, the biasing member biases the distal body to retract the syringe within the shield.

The various embodiments of the autoinjector discussed herein provide numerous advantages over conventional autoinjector devices. For example, the present autoinjector uses a rotating drum e.g., activator 302, that interfaces with several components via tracks, stops, and cams, to control the sequence of operational states during use. This provides several benefits over conventional autoinjector mechanisms, especially in a marketplace looking for premium features on a low-cost disposable device.

The primary advantage of using a rotating drum is that it allows greater control of the device operation and features, without adding undue complexity and cost. The rotating drum provides for a highly configurable sequence of events with precise control of each operational state. The rotating drum is also able to integrate features that would be impractical and/or expensive with conventional autoinjector mechanisms. Certain advantageous features integrated into the rotating drum concept include auto-retraction, locked syringe positions, no-force shield, and status indicators.

Another advantage of the present autoinjector embodiments is auto retraction of the needle into the autoinjector body. In contrast, conventional autoinjectors use a spring loaded shield to cover the needle as the device is removed from the injection site. Thus, in combination with status indicators and no-force shield, auto retraction simplifies operational steps required of the user in order to ensure an effective and safe injection.

A further advantage of the present autoinjector embodiments is its locked syringe positions. Typical conventional autoinjectors have mechanisms that do not allow for securely locking the syringe in position, and instead rely on spring forces to keep it in position during handling and after usage. This may allow the syringe and needle to move outwardly during shocks or mishandling, possibly causing needle stick injuries. In contrast, the rotating drum of the present embodiments allows for a securely locked needle in all positions.

Another advantage of the present autoinjector embodiments is its no-force shield. Typical conventional autoinjectors require the user to maintain force against the injection site during injection to keep the needle shield's spring compressed until the injection is complete, at which time the pressure is removed and the shield extends and locks in place as the device is withdrawn. This is supposed to be a simple and effective approach, but the user's grip may slip or be repositioned while the injection is occurring. As a result, the device may be pushed away from the injection site by the shield's spring, thus locking the shield over the needle and injecting the remaining contents into air. This results in an underdose which is not only a nuisance but in some cases can result in a fatal error. The present design features the instant invention eliminates this possibility since the shield component is only required to travel a minimal distance to initiate an injection, and does not require a return force since the retraction occurs independently.

A further advantage of the present autoinjector embodiments is its status indicators. An advantage of the rotating drum approach is that it allows for a straightforward way to communicate the current state of the autoinjector's operation, such as “Ready to activate,” “Injection in progress,” and “Injection completed.” In conventional autoinjector devices where the sequencing elements are non-centralized, e.g., spread out across the device, it becomes challenging to display the device's state conveniently in one place when there are more than two operational states. With the design of the present embodiments, components such as the shield, syringe, and actuator have physical interfaces with the rotating drum so that the drum itself can be used to indicate to the user the current state of operation in a convenient and straightforward way, using e.g., windows and visible markings, or switches and electronic indicators.

It will be appreciated by those skilled in the art that changes could be made to the preferred embodiments described above without departing from the broad inventive concept thereof. For example, additional components, types of flow regulators, or visual indicators can be added or used with the autoinjector. It is to be understood, therefore, that this invention is not limited to the particular embodiments disclosed, but it is intended to cover modifications within the spirit and scope of the present invention as defined by the claims.

What is claimed is:

1. An autoinjector comprising:
a canister having a liquefied gas;
a flow regulator for engaging the canister to receive a flow of gas generated by the liquefied gas; and
an actuator coupled to the canister and the flow regulator, wherein the actuator includes a passageway for receiving the flow of gas downstream from the flow regulator, and
wherein the actuator moves relative to the canister between an engaged position whereby the flow regulator is in fluid communication with the canister for receiving the flow of gas and a storage position whereby the flow regulator is spaced from the canister.

2. The autoinjector of claim 1, wherein actuator further includes an exhaust passageway about a distal end of the actuator.

3. The autoinjector of claim 2, further comprising a seal sealingly engaging the exhaust passageway when the actuator is in both the engaged and storage positions.

4. The autoinjector of claim 1, further comprising a seal between the canister and the flow regulator.

5. The autoinjector of claim 4, wherein the seal is between the canister and the actuator.

6. The autoinjector of claim 1, wherein the flow regulator includes a piercing member for engaging the canister.

7. The autoinjector of claim 1, wherein the flow regulator includes a porous member for receiving the flow of gas.

8. The autoinjector of claim 1, wherein the flow regulator includes:
a body;
a piercing member extending from the body for piercing the canister;
a passageway through the body for receiving the flow of gas; and
a porous member covering the passageway for controlling the flow of gas through the passageway.

9. The autoinjector of claim 1, wherein the flow regulator includes:
a body having a central through hole;
a porous member extending across the through hole about a proximal end of the body; and
a piercing member extending from the porous member.

10. The autoinjector of claim 1, wherein the flow regulator includes:
a body;
a piercing member extending from the body for piercing the canister;
a passageway through the body for receiving the flow of gas; and
a porous pellet blocking an end of the passageway for controlling the flow of gas through the passageway.

11. An autoinjector comprising:
a canister having a liquefied gas for delivering a flow of gas; and
an actuator coupled to the canister, the actuator including:
a housing body,
a through hole through the housing body for receiving the flow of gas, and
a flow regulation device adjacent the through hole,
wherein the actuator moves relative to the canister between an engaged position whereby the canister engages the actuator to allow fluid communication of the flow of gas with the through hole, and a storage position whereby the canister is disengaged with the actuator and the through hole is not in fluid communication with the flow of gas.

12. The autoinjector of claim 11, wherein the actuator includes a hub assembly having a piercing member, and wherein the hub assembly engages the canister in the engaged position and is spaced from the canister in the disengaged position.

13. The autoinjector of claim 11, wherein the flow regulation device includes a porous member for receiving the flow of gas.

14. The autoinjector of claim 13, wherein the porous member is a disc, a pellet or a wall portion.

15. The autoinjector of claim 13, wherein the flow regulation device is formed from a metal.

16. An autoinjector comprising:
a shield housing a syringe moveable relative to the shield; and
an actuator assembly for delivering a driving force that operatively engages the syringe, the actuator assembly including:
an actuator,
an activator coupled to the actuator and movable relative to the actuator between a first position wherein the activator is stationary with respect to the actuator, a second position circumferentially spaced from the first position, and a third position circumferentially spaced from the first and second positions, and
a biasing member biasing the activator and actuator relative to each other to move one of the activator and actuator between first, second and third positions.

17. The autoinjector of claim 16, wherein the second position is circumferential spaced from the first position in a first direction, and the third position is circumferential spaced from the first position in the first direction and axially spaced from the second position.

18. The autoinjector of claim 16, wherein the second position is axially spaced from the first position.

19. The autoinjector of claim 16, wherein the biasing member biases one of the actuator and activator in both an axial direction and a rotational direction.

20. The autoinjector of claim 16, wherein the activator further includes an activator cam surface,
wherein the shield includes a proximally extending latch member, and
wherein the shield moves between an initial position covering a distal end of the syringe and an injection position, and when moving from the initial position to the injection position the proximally extending latch member engages the activator cam surface to move the activator from the first position to the second position.
